(19) United States
(12) Patent Application Publication
(10) Pub. No.: US 2025/0375502 A1
(43) Pub. Date: Dec. 11, 2025

(54) COMBINATION OF IL-12 AND OX40L FOR CANCER IMMUNOTHERAPY

(71) Applicant: WUHAN HOUXIAN BIOPHARMACEUTICAL CO. LTD., Wuhan, Hubei (CN)

(72) Inventors: Hongbing SHU, Wuhan, Hubei (CN); Mingming HU, Wuhan, Hubei (CN); Shu LI, Wuhan, Hubei (CN); Mi LI, Wuhan, Hubei (CN); Qing YANG, Wuhan, Hubei (CN)

(21) Appl. No.: 18/877,126

(22) PCT Filed: Jun. 21, 2023
(86) PCT No.: PCT/CN2023/101597

§ 371 Date: Dec. 19, 2024

(30) Foreign Application Priority Data
Jun. 22, 2022 (WO) PCT/CN2022/100453

(51) Int. Cl. A61K 38/20; A61K 38/19; A61K 39/00; A61K 39/395; A61P 35/00

(52) U.S. Cl. CPC A61K 38/208; A61K 38/191; A61K 38/193; A61K 39/3955; A61P 35/00; A61K 2039/505

(57) ABSTRACT

Provided are compositions and methods for treating cancers. It is demonstrated herein that mRNA molecules expressing an OX40 agonist protein and the IL-12 protein, when used in combination, achieve synergistic anti-tumor effects. Such synergistic effect is further enhanced when a soluble portion of the OX40 ligand (OX40L) is used as the agonist, instead of the full-length OX40L protein. The mRNA molecules are preferably synthetic and packaged in lipid nanoparticles for delivery. Whether delivered through intratumoral injections or injected by other routes, these mRNA molecules can effectively inhibit tumor growth at local as well as distal sites. In addition, with the increased anti-tumor efficacy, the combinations, in particular at a mass ratio of IL-12 to OX40L between 1:1 and 1:3, are associated with reduced toxicity. Interestingly, when GM-CSF is further added to the combination, the anti-tumor effects are further improved.

### BACKGROUND

The generation of immunity to cancer is a cyclic process that can be self-propagating, leading to an accumulation of immune-stimulatory factors that in principle should amplify and broaden T cell responses. The cycle is also characterized by inhibitory factors that lead to immune regulatory feedback mechanisms, which can halt the development or limit the immunity.

This cycle can be divided into a few major steps, starting with the release of antigens from the cancer cell and ending with the killing of cancer cells. The cancer antigen is presented by dendritic cells or antigen presenting cells (APCs), followed by priming and activation of APCs and T cells. Then, trafficking of the activated T cells to the tumor is followed by T cell infiltration into the tumor tissue, and T cell recognition of tumor cells, leading to killing of the tumor cells.

Each step of the Cancer-Immunity Cycle requires the coordination of numerous factors, both stimulatory and inhibitory in nature. Stimulatory factors promote immunity, whereas inhibitors help keep the process in check and reduce immune activity and/or prevent autoimmunity. Immune checkpoint proteins, such as CTLA4, can inhibit the development of an active immune response by acting primarily at the level of T cell development and proliferation. Immune checkpoint proteins are distinguished from immune rheostat (“immunostat”) factors, such as PD-L1, which can have an inhibitory function that primarily acts to modulate active immune responses in the tumor bed.

Cancer immunotherapies, such as checkpoint inhibitors and adoptive cell therapy, manipulate the immune system to recognize and attack cancer cells. An example is to enhance the effector function of tumor-specific Teff cells, and another is to reduce the inhibitory function of tumor-specific Treg cells.

A typical cancer immunotherapy uses an antibody that targets a checkpoint protein or a tumor-associated antigen, or the immune cells engineered to express a targeting receptor. Antibodies, however, generally have short half-lives, and cell therapies are extremely expensive to manufacture.

### SUMMARY

It is demonstrated herein that mRNA molecules expressing the OX40L protein and the IL-12 protein, when used in combination, achieved synergistic anti-tumor effects. Such synergistic effect was further enhanced when a soluble portion of the OX40 ligand (OX40L) was used as the agonist, instead of the full-length OX40L protein. The mRNA molecules are preferably synthetic and packaged in lipid nanoparticles for delivery. Whether delivered through intratumoral injections or injected by other routes, these mRNA molecules can effectively inhibit tumor growth at local as well as distal sites. In addition, with the increased anti-tumor efficacy, the combinations, in particular at a mass ratio of IL-12 to OX40L between 1:1 and 1:3, are associated with reduced toxicity. Interestingly, when GM-CSF was further added to the combination, the anti-tumor effects were further improved.

Accordingly, one embodiment of the present disclosure provides a method for treating cancer in a patient, comprising administering to the patient a first mRNA encoding an OX40 agonist, and a second mRNA encoding IL-12, wherein the OX40 agonist is an OX40 ligand (OX40L), a polypeptide comprising the extracellular domain of OX40L, or an agonist anti-OX40 antibody or antigen-binding fragment thereof.

In some embodiments, the first mRNA and the second mRNA are included in the same RNA molecule. In some embodiments, the RNA molecule encodes a polypeptide that comprises the OX40 agonist and IL-12 as a fusion protein, or encodes separate polypeptides. In some embodiments, the first mRNA and the second mRNA are separate molecules.

In some embodiments, the OX40 agonist is the OX40L. In some embodiments, the OX40L comprises the amino acid sequence of SEQ ID NO:1 or 2, or an amino acid sequence having at least 85% sequence identity to SEQ ID NO:1 or 2. In some embodiments, the first mRNA comprises the nucleic acid sequence of SEQ ID NO:3 or 4, or an nucleic acid sequence having at least 85% sequence identity to SEQ ID NO:3 or 4.

In some embodiments, the extracellular domain of OX40L comprises amino acid residues 52-183 of SEQ ID NO:1, or a sequence having at least 85% sequence identity to amino acid residues 52-183 of SEQ ID NO:1. In some embodiments, the polypeptide further comprises an oligomerization domain or a transmembrane domain. In some embodiments, the OX40 agonist comprises a Fc domain fused to the extracellular domain of OX40L, and wherein the ( ) 40 agonist is a soluble protein not containing the transmembrane domain of the OX40L protein.

In some embodiments, the second mRNA encodes IL-12A. In some embodiments, the IL-12A comprises an amino acid sequence selected from the group consisting of residues 57-253 of SEQ ID NO:5, residues 57-239 of SEQ ID NO:6, residues 57-215 of SEQ ID NO:7 and residues 23-219 of SEQ ID NO:8, or an amino acid having at least 85% sequence identity to any amino acid sequence of the group. In some embodiments, the second mRNA comprises the nucleic acid sequence of SEQ ID NO:9, 10, 11 or 12, or a nucleic acid sequence having at least 85% sequence identity to SEQ ID NO:9, 10, 11 or 12.

In some embodiments, the method further comprises administering to the patient a third mRNA, wherein the third mRNA encodes residues 23-328 of SEQ ID NO:13.

In some embodiments, the second mRNA encodes IL-12B. In some embodiments, the IL-12B comprises residues 23-328 of SEQ ID NO:13, or an amino acid sequence having at least 85% sequence identify to residues 23-328 SEQ ID NO:13. In some embodiments, the second mRNA comprises the nucleic acid sequence of SEQ ID NO; 14, or a nucleic acid sequence having at least 85% sequence identity to SEQ ID NO:14.

In some embodiments, the first mRNA and the second mRNA are administered at a mass ratio of 4:1 to 0.5:1. In some embodiments, the first mRNA and the second mRNA are administered at a mass ratio of 3:1 to 0.75:1. In some embodiments, the first mRNA and the second mRNA are administered at a mass ratio of 2:1 to 0.9:1.

In some embodiments, the method further comprises administering to the patient a fourth mRNA encoding GM-CSF (granulocyte-macrophage colony-stimulating factor). In some embodiments, the method further comprises administering to the patient a fourth mRNA encoding TNFR (tumor necrosis factor receptor). In some embodiments, the method further comprises administering to the patient a fourth mRNA encoding GSDMD (Gasdermin D).

In some embodiments, the method does not include administration of an immune checkpoint inhibitor, an interferon, another IL-12 family member, or another cytokine, or a nucleic acid encoding therefor. In some embodiments, the immune checkpoint inhibitor is PD-1, PD-L1 or CTLA-4 inhibitor. In some embodiments, the interferon is IFN-α, IFN-β, or IFN-γ. In some embodiments, the other IL-12 family members comprise IL-23, IL-27 and IL-35. In some embodiments, the other cytokine is IL-18.

Also provided, in another embodiments, is a method for treating cancer in a patient, comprising administering to the patient a first agent comprising a mRNA encoding IL-12, and a second agent comprising an OX40 agonist, wherein the OX40 agonist is an agonist anti-OX40 antibody or antigen-binding fragment thereof, an OX40 ligand (OX40L), or a polypeptide comprising the extracellular domain of OX40L. In some embodiments, the OX40 agonist is an agonist anti-OX40 antibody.

In some embodiments, each mRNA is a linear mRNA or circular mRNA. In some embodiments, each mRNA further comprises a miRNA binding site. In some embodiments, each mRNA does not include chemical modification that reduces immunogenicity. In some embodiments, the mRNA does not include chemical modification to the backbone. In some embodiments, each mRNA only includes natural nucleosides.

In some embodiments, at least one of the uridine nucleosides in the mRNAs are chemically modified. In some embodiments, the chemically modified uridine nucleosides are N1-methylpseudouridines. In some embodiments, the first mRNA and the second mRNA are formulated with a pharmaceutically acceptable carrier.

In some embodiments, the carrier comprises a lipid nanoparticle (LNP). In some embodiments, the LNP comprises (a) a molar ratio of 40-60% ionizable amino lipid, a molar ratio of 8-16% phospholipid, a molar ratio of 30-45% sterol, and a molar ratio of 1-5% PEG-modified lipid, (b) a molar ratio of 45-65% ionizable amino 40 lipid, a molar ratio of 5-10% phospholipid, a molar ratio of 25-40% sterol, and a molar ratio of 0.5-5% PEG modified lipid, (c) a molar ratio of 40-60% ionizable amino lipid, a molar ratio of 8-16% phospholipid, a molar ratio of 30-45% sterol, and a molar ratio of 1-5% PEG modified lipid, (d) a molar ratio of 45-65% ionizable amino lipid, a molar ratio of 5-10% phospholipid, a molar ratio of 25-40% sterol, and a molar ratio of 0.5-5% PEG modified lipid, (e) a molar ratio of 40-60% ionizable amino lipid, a molar ratio of 8-16% phospholipid, a molar ratio of 30-45% sterol, and a molar ratio of 1-5% PEG modified lipid, or (f) a molar ratio of 45-65% ionizable amino lipid, a molar ratio of 5-10% phospholipid, a molar ratio of 25-40% sterol, and a molar ratio of 0.5-5% PEG modified lipid.

In some embodiments, each mRNA is packaged in a liposome. In some embodiments, the liposome comprises a cationic lipid, a non-cationic lipid, a cholesterol-based lipid and a PEG modified lipid.

In some embodiments, the cationic lipid is selected from the group consisting of 1,1′-((2-(4-(2-((2-(bis(2-hydroxydodecyl)amino)ethyl) 2-hydroxydodecyl)amino)ethyl)piperazin-1-yl)ethyl)azanediyl)bis(dodecan-2-ol) (C12-200), (6Z,9Z,28Z,31Z)-heptatriaconta-6,9,28,31-tetraen-19-yl 4-(dimethylamino) butanoate (MC3), N,N-dimethyl-2,3-bis((9Z,12Z)-octadeca-9,12-dien-1-yloxy) propan-1-amine (DLinDMA), 2-(2,2-di((9Z,12Z)-octadeca-9,12-dien-1-yl)-1,3-dioxolan-4-yl)-N,N-dimethylethanamine (DLinKC2DMA, [XTC2]), 3,6-bis(4-(bis(2-hydroxydodecyl)amino)butyl)piperazine-2,5-dione (cKK-E12), 10,13-dimethyl-17-(6-methylheptan-2-yl)-2,3,4,7,8,9,10,11,12,13,14,15,16,17-tetradecahydro-1H-cyclopenta[a]phenanthren-3-yl 3-(1H-imidazol-5-yl) propanoate (ICE), (15Z,18Z)-N,N-dimethyl-6-((9Z,12Z)-octadeca-9,12-dien-1-yl)tetracosa-15,18-dien-1-amine (HGT5000), (4Z,15Z,18Z)-N,N-dimethyl-6-((9Z,12Z)-octadeca-9,12-dien-1-yl)tetracosa-4,15,18-trien-1-amine (HGT5001), N,N-dioleyl-N,N-dimethylammonium chloride (DODAC), N,N-distearyl-N,N-dimethylammonium bromide (DDAB), 1,2-dimyristyloxyproyl-3-dimethyl-hydroxyethyl ammonium bromide (DMRIE), dioleoyloxy-N-[2-sperminecarboxamido)ethyl]-N,N-dimethyl-1-propaniminiumtrifluoroacetate (DOSPA), dioctadecylamidoglycyl spermine (DOGS), 1,2-diolcoyl-3-dimethylammonium-propane (DODAP), N,N-dimethyl-(2,3-diolcyloxy) propylamine (DODMA) and N,N-dimethyl-(2,3-dimyristyloxy) propylaminc (DMDMA), 1,2-dilinolenyloxy-N,N-dimethylaminopropane (DLenDMA), (2S)-2-(4-((10,13-dimethyl-17-(6-methylheptan-2-yl)-2,3,4,7,8,9,10,11,12,13,14,15,16,17-tetradecahydro-1H-cyclopenta[a]phenanthren-3-yl)oxy) butoxy)-N,N-dimethyl-3-((9Z,12Z)-octadeca-9,12-dien-1-yloxy) propan-1-amine (CLinDMA), 2-[5′-(cholest-5-en-3 [betal]-oxy)-3′-oxapentoxy)-3-dimethyl-1-1 (cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), (9Z,9′Z,12Z,12′Z)-3-(dimethylamino) propane-1,2-diyl bis(octadeca-9,12-dienoate) (DLinDAP), 1,2-dilinolcylcarbamyl-3-dimethylaminopropane (DLinCDAP), 2,2-dilinoleyl-4-dimethylaminomethyl-[1,3]-dioxolane (DLin-K-DMA), 2-((2,3-bis((9Z,12Z)-octadeca-9,12-dien-1-yloxy) propyl)disulfanyl)-N,N-dimethylethanamine (HGT4003), and

In some embodiments, the cholesterol-based lipid is cholesterol or PEGylated cholesterol. In some embodiments, the cationic lipid constitutes about 30-50% of the liposome by molar ratio. In some embodiments, the ratio of cationic lipid:non-cationic lipid:cholesterollipid:PEGylated lipid is approximately 40:30:25:5 by molar ratio. In some embodiments, the liposome comprises a combination selected from the group consisting of: cKK-E12, 1,2-dioleyl-sn-glycero-3-phosphoethanolamine (DOPE), cholesterol and 1,2-dimyristoyl-sn-glycerol, methoxypolyethylene Glycol (DMG-PEG2K); C12-200, DOPE, cholesterol and DMG-PEG2K; HGT4003, DOPE, cholesterol and DMG-PEG2K; or ICE, DOPE, cholesterol and DMG-PEG2K.

In some embodiments, the administration is subcutaneous injection, intramuscular injection, intraperitoneal injection, thoracic injection, intravenous injection, arterial injection, or a combination thereof.

In some embodiments, the administration is made at a frequency of 3 times a week, twice a week, once a week, once every 2 weeks, once every 3 weeks, once every 4 weeks, once a month, or once every 3-6 months.

In some embodiments, the cancer is selected from the group consisting of squamous cell carcinoma, lung cancer, peritoneal cancer, hepatocellular carcinoma, gastric cancer, pancreatic cancer, glioblastoma, cervical cancer, ovarian cancer, liver cancer, bladder cancer, urethral cancer, breast cancer, colon cancer, rectal cancer, colorectal cancer, endometrial cancer, uterine cancer, salivary gland cancer, kidney cancer, prostate cancer, vulvar cancer, thyroid cancer, liver cancer, anal cancer, soft tissue sarcoma, neuroblastoma, penile cancer, melanoma, superficial spreading melanoma, lentigines melanoma, acral melanoma, nodular melanoma, multiple bone marrow tumor, B-cell lymphoma, chronic lymphocytic leukemia, non-Hodgkin's lymphoma, acute lymphoblastic leukemia, hairy cell leukemia, chronic myeloblastic leukemia, post-transplant lymphoproliferative disorder, brain tumor, and brain cancer and head and neck cancer, preferably colon cancer, breast cancer and lung cancer.

Also provided are compositions useful for carrying out the disclosed methods. In one embodiment, a pharmaceutical composition is provided comprising a first mRNA encoding an OX40 agonist, and a second mRNA encoding IL-12, wherein the OX40 agonist is an OX40 ligand (OX40L), a polypeptide comprising the extracellular domain of OX40L or an agonist anti-OX40 antibody or antigen-binding fragment thereof.

Another embodiment provides a pharmaceutical composition comprising a first agent comprising a mRNA encoding IL-12, and a second agent comprising an OX40 agonist, wherein the OX40 agonist is an agonist anti-OX40 antibody or antigen-binding fragment thereof, an OX40 ligand (OX40L), or a polypeptide comprising the extracellular domain of OX40L.

### BRIEF DESCRIPTION OF THE DRAWINGS

FIG. 1 shows the results of screening for single or combination mRNA molecules having anti-tumor effects in a colon cancer animal model.

FIG. 2 shows that the therapeutic effects of the combination of OX40L and IL-12 mRNA spread from the injection side to the distal tumor side.

FIG. 3 shows that both intratumoral injection and distal subcutaneous injections of the OX40L and IL-12 mRNA led to significant inhibition of tumor growth.

FIG. 4 shows the anti-tumor effects of OX40L and IL-12 mRNA in a lung cancer model.

FIG. 5 shows that, in the lung cancer model, the therapeutic effects of the combination of OX40L and IL-12 mRNA spread from the injection side to the distal tumor side.

FIG. 6 shows the anti-tumor effects of OX40L and IL-12 mRNA in a triple-negative breast cancer model.

FIG. 7 shows that, in the breast cancer model, the therapeutic effects of the combination of OX40L and IL-12 mRNA spread from the injection side to the distal tumor side.

FIG. 8 shows that human OX40L and IL-12 mRNAs were effective in treating breast cancer in Tupaia belangeri.

FIG. 9 shows tumor mass reduction in animals following intratumoral injection of mRNA expressing the test agents.

FIG. 10 shows overall body weight changes in animals following intratumoral injection of mRNA expressing the test agents.

FIG. 11 shows tumor volumes at the injected side, on days 8, 11, 14 and 17 post-implantation, after two intratumoral injections of the indicated mRNAs at the indicated dosages.

FIG. 12 shows tumor volumes at the distal side, on days 8, 11, 14 and 17 post-implantation, after two intratumoral injections of the indicated mRNAs at the indicated dosages.

FIG. 13 shows tumor mass reduction in animals following intratumoral injection of mRNA expressing IL-12 and a test antibody.

FIG. 14 shows overall body weight changes in animals following intratumoral injection of the indicated mRNA/antibody.

FIG. 15 shows tumor volumes at the injected side, on days 8, 11, 14 and 17 post-implantation, after two intratumoral injections of the indicated mRNA/antibody.

FIG. 16 shows tumor volumes at the distal side, on days 8, 11, 14 and 17 post-implantation, after two intratumoral injections of the indicated mRNA/antibody.

FIG. 17 shows tumor mass reduction in animals following intratumoral injection of test mRNA molecules or their combinations.

FIG. 18 shows overall body weight changes in animals following intratumoral injection of the indicated mRNA.

FIG. 19 shows tumor volumes at the injected side, on days 9, 12, 15 and 18 post-implantation, after two intratumoral injections of the indicated mRNA.

FIG. 20 shows tumor volumes at the distal side, on days 9, 12, 15 and 18 post-implantation, after two intratumoral injections of the indicated mRNA.

FIG. 21 shows tumor mass reduction in animals following intratumoral injection of test mRNA molecules or their combinations.

FIG. 22 shows overall body weight changes in animals following intratumoral injection of the indicated mRNA.

FIG. 23 shows tumor volumes at the injected side, on days 9, 12, 15 and 18 post-implantation, after two intratumoral injections of the indicated mRNA.

FIG. 24 shows tumor volumes at the distal side, on days 9, 12, 15 and 18 post-implantation, after two intratumoral injections of the indicated mRNA.

FIG. 25 shows tumor mass reduction in animals following intratumoral injection of test mRNA molecules at the indicated doses.

FIG. 26 shows overall body weight changes in animals following intratumoral injection of the indicated mRNA.

FIG. 27 shows tumor volumes at the injected side, on days 8, 11, 14 and 17 post-implantation, after two intratumoral injections of mRNA at the indicated doses.

FIG. 28 shows tumor volumes at the distal side, on days 8, 11, 14 and 17 post-implantation, after two intratumoral injections of mRNA at the indicated doses.

### DETAILED DESCRIPTION

### Definitions

It is to be noted that the term “a” or “an” entity refers to one or more of that entity; for example, “an mRNA,” is understood to represent one or more mRNA. As such, the terms “a” (or “an”), “one or more,” and “at least one” can be used interchangeably herein.

OX40, also known as CD134 and tumor necrosis factor receptor superfamily, member 4 (TNFRSF4), is a member of the TNFR-superfamily of receptors. Unlike CD28 which is constitutively expressed on resting naïve T cells, OX40 is a secondary co-stimulatory immune checkpoint molecule, expressed after 24 to 72 hours following activation.

OX40L, also known as CD252, is the ligand for OX40 and is stably expressed on many antigen-presenting cells such as DC2s (a subtype of dendritic cells), macrophages, and activated B lymphocytes. OX40L is also present on the surface of many non-immune cells, such as the endothelial cells and the smooth muscle cells. The surface expression of OX40L can be induced by many pro-inflammatory mediators, such as TNF-α, IFN-γ, and PGE2 (Prostaglandin E2).

A representative nucleic acid sequence for human OX40L (isoform 1) is provided in NCBI Reference No. NM_003326 with a corresponding protein sequence in NP_003317. Another representative nucleic acid sequence for human OX40L (isoform 2) is provided in NCBI Reference No. NM_001297562 with a corresponding protein sequence in NP 001284491. Isoform 1 has a longer N-terminus than isoform 2, but otherwise they are identical.

Interleukin 12 (IL-12) is an interleukin that is naturally produced by dendritic cells, macrophages, neutrophils, and human B-lymphoblastoid cells (NC-37) in response to antigenic stimulation. IL12 is a heterodimeric cytokine encoded by two separate genes, IL12A (p35) and IL12B (p40).

IL-12 is involved in the differentiation of naive T cells into Th1 cells. It stimulates the production of interferon-gamma (IFN-γ) and tumor necrosis factor-alpha (TNF-α) from T cells and natural killer (NK) cells, and reduces IL-4 mediated suppression of IFN-γ. IL-12 plays an important role in the activities of natural killer cells and T lymphocytes. IL-12 mediates enhancement of the cytotoxic activity of NK cells and CD8+ cytotoxic T lymphocytes.

IL-12 binds to the IL-12 receptor, which is a heterodimeric receptor formed by IL-12Rβ1 and IL-12Rβ2. Upon binding, IL-12R-β2 becomes tyrosine phosphorylated and provides binding sites for kinases, Tyk2 and Jak2.

A representative nucleic acid sequence for human IL-12A (isoform 1) is provided in NCBI Reference No. NM_000882 with a corresponding protein sequence in NP_000873. Another representative nucleic acid sequence for human IL-12A (isoform 2) is provided in NCBI Reference No. NM_001354582 with a corresponding protein sequence in NP_001341511. Another representative nucleic acid sequence for human IL-12A (isoform 3) is provided in NCBI Reference No. NM_001354583 with a corresponding protein sequence in NP_001341512. Another representative nucleic acid sequence for human IL-12A (isoform 4) is provided in NCBI Reference No. NM_001397992 with a corresponding protein sequence in NP_001384921.

A representative nucleic acid sequence for human IL-12B is provided in NCBI Reference No. NM_002187 with a corresponding protein sequence in NP_002178.

Granulocyte-macrophage colony-stimulating factor (GM-CSF), also known as colony stimulating factor 2 (CSF2), is a monomeric glycoprotein secreted by macrophages, T cells, mast cells, NK cells, endothelial cells and fibroblasts that functions as a cytokine. GM-CSF stimulates stem cells to produce granulocytes (neutrophils, eosinophils, and basophils) and monocytes. Monocytes exit the circulation and migrate into tissue, whereupon they mature into macrophages and dendritic cells. Thus, it is part of the immune inflammatory cascade, by which activation of a small number of macrophages can rapidly lead to an increase in their numbers, a process crucial for fighting infection. GM-CSF also has some effects on mature cells of the immune system. These include, for example, inhibiting neutrophil migration and causing an alteration of the receptors expressed on the cells surface.

A representative nucleic acid sequence for human GM-CSF is provided in NCBI Reference No. NM_000758 with a corresponding protein sequence in NP_000749.

Combination of OX40L and IL-12 mRNA

Cancer immunotherapies have shown great promises by using small molecules, antibodies or engineered immune cells targeting numerous factors involved in the cancer-immunity cycle. A typical strategy involves activation of stimulatory factors that promote immunity, or inhibition of factors that reduce immune activity and/or prevent autoimmunity. Some prominent examples are anti-CTLA4 antibodies and anti-PD-1 or anti-PD-L1 antibodies.

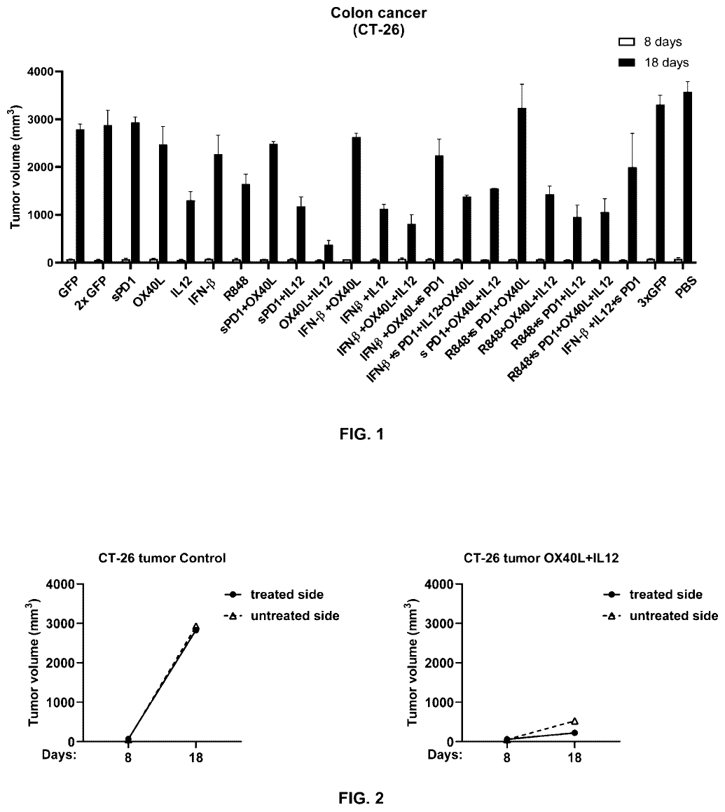

Delivery of a cancer therapeutic through an encoding mRNA is an emerging technology, which has shown some promises. There are some unforeseen obstacles, however. For instance, when an mRNA encoding a soluble PD1 fragment (sPD1) which is a known PD-1/PD-L1 inhibitor was delivered intratumorally, it exhibited no inhibition of tumor growth at all (Example 1, FIG. 1).

Also, intratumoral injection of the mRNA encoding OX40L only exhibited slight inhibition of tumor growth. By contrast, intratumoral injection of the mRNA encoding IL-12 led to marked tumor growth inhibition, at about 50% rates. (FIG. 1). Quite unexpectedly, when both mRNA were delivered to the same animal, the inhibition reached a whopping ˜90%. Such significantly enhanced therapeutic efficacy clearly indicates synergism between these molecules.

This result is unexpected in particular in view that the addition of other seemingly therapeutic mRNA molecule did not further increase efficacy. For instance, despite its moderate anti-tumor effect as a stimulator of innate immunity, the single small molecule agent R848 (a ligand for Toll-like receptor 7/8) actually decreased other agents' anti-tumor effects when used in combinations (FIG. 1).

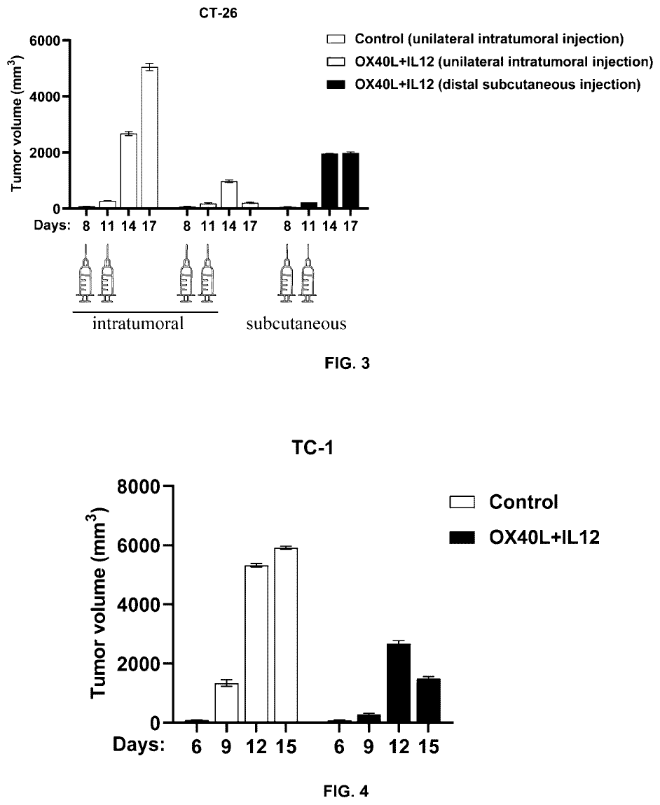

Additional experimental data presented in the examples further reinforce the therapeutic efficacy of these combination approach. As shown in FIG. 2, not only did intratumoral injection inhibit local tumor growth, but it also achieved similar magnitude of therapeutic efficacy at distal regions, across the tumor block. Then, as demonstrated in FIG. 3, even subcutaneous injections at distal body sites also resulted in potent therapeutic effects.

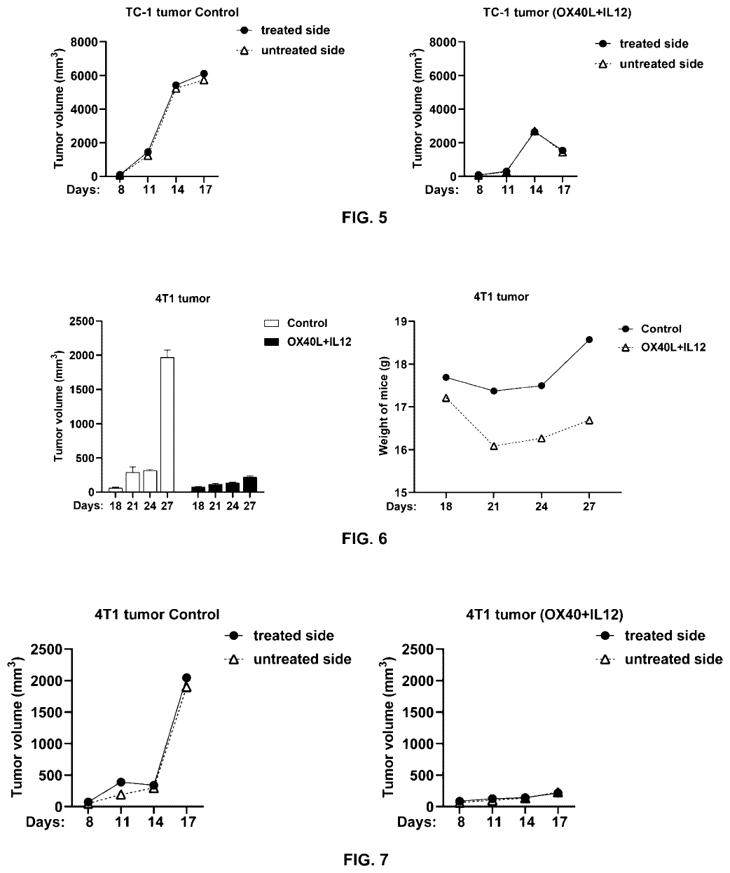

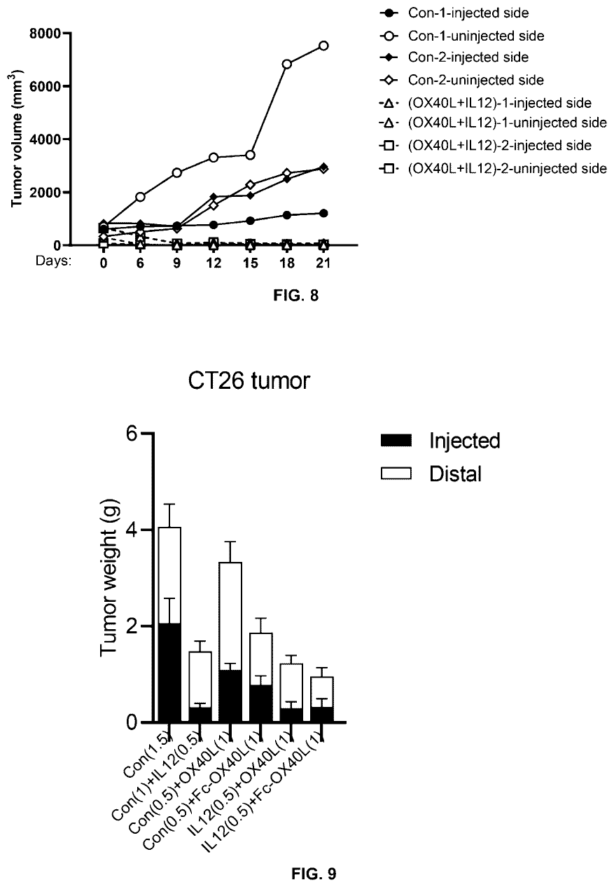

Moreover, the efficacy of this combination was tested in multiple cancer types, including colon cancer (FIG. 1-3), lung cancer (FIG. 4-5), and breast cancer (FIG. 6-7). Also, the actual human mRNA sequences were tested in a Tupaia belangeri breast cancer model (FIG. 8). Therefore, the present data presents a new therapeutic regime for multiple cancer types.

In another unexpected discovery, when a soluble counterpart of the OX40L protein was used in the combination, further improvement of the therapeutic efficacy was observed (Example 7, FIG. 9-12), in particular at the distal side of the animal, from the injection side (FIGS. 9 and 12). This soluble counterpart was an extracellular fragment of the OX40L protein fused to an IgG Fc fragment (Fc-OX40L).

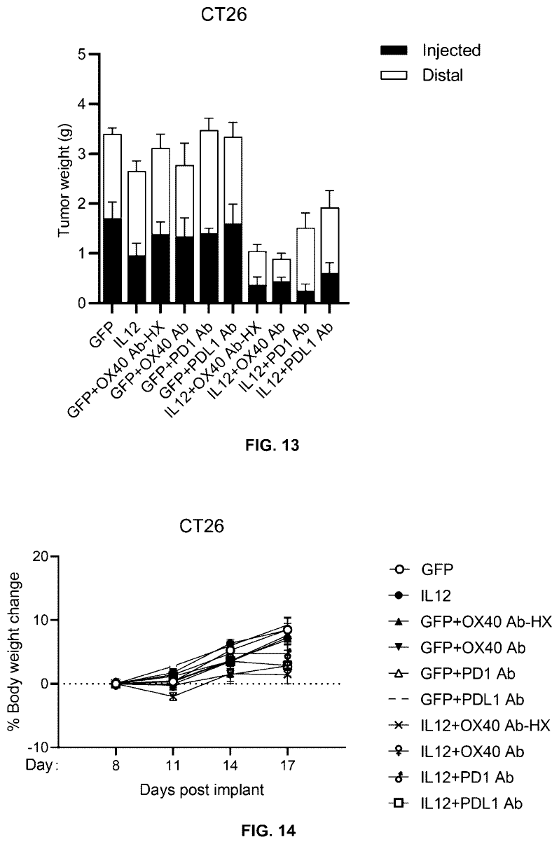

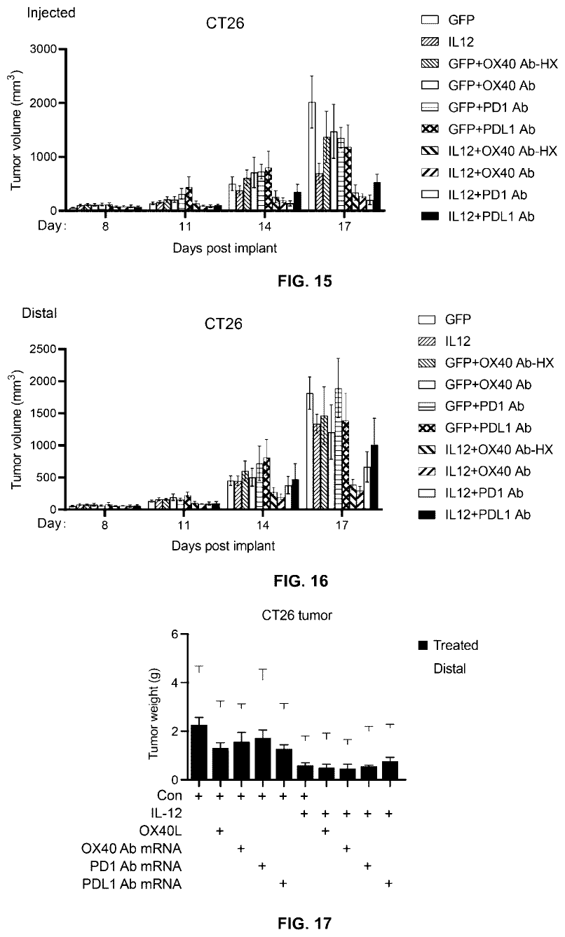

Moreover, as demonstrated in Examples 8 and 9 and FIG. 13-20, the agonist effect of OX40L can be substituted with an agonist antibody while achievable comparable results. The antibody can be delivered as a protein directly to the patient, or expressed in vivo following delivery of an encoding mRNA.

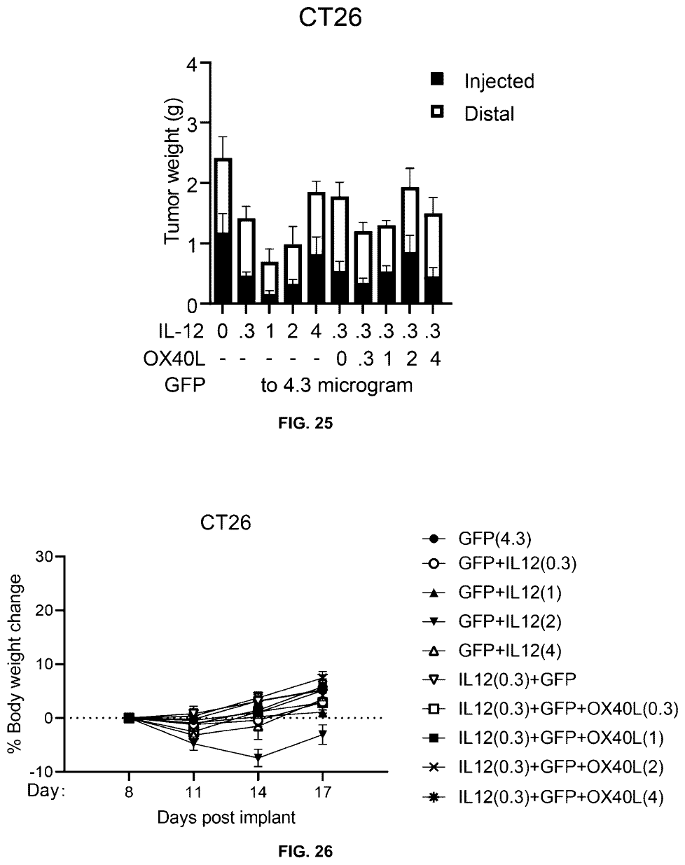

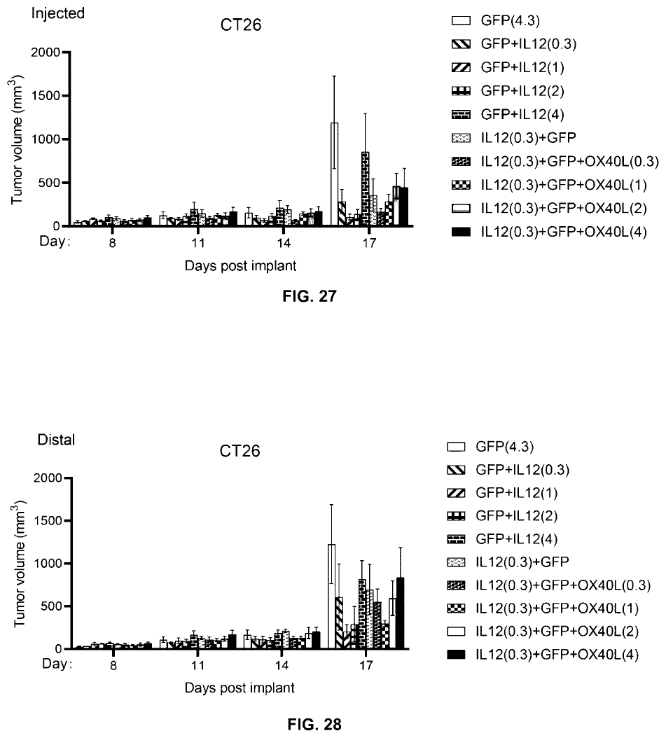

In addition, with the increased anti-tumor efficacy, the combinations also led to reduced toxicity. For instance, as shown in FIG. 25, a 0.3 μg IL-12/0.3 μg OX40L combination resulted in similar tumor inhibition efficacy as 2.0 μg IL-12 alone. The solo 2.0 μg IL-12 treatment, however, led to significant body weight reduction (lowest curve in FIG. 26). Even at 1 μg, the IL-12 alone treatment inhibited body weight growth altogether (second lowest curve in FIG. 26). From these experiments in Example 11, an optimal mass ratio between IL-12 and OX40L was obtained, at about 1:1 to 1:3.

In another interesting finding, when GM-CSF was further added to the combination, the anti-tumor effects were further improved. The magnitude of improvement by GM-CSF was greater than by GSDMD and TNFR, two other commonly used immune modulators in cancer therapies, surprisingly.

According to one embodiment of the present disclosure, therefore, provided is a method for treating cancer that entails administration of a mRNA encoding an OX40 agonist (e.g., OX40L or a protein/polypeptide/antibody or their combination that activates OX40 (e.g., an OX40L protein or anti-OX40 agonist antibody)), and an mRNA encoding IL-12 or a protein polypeptide/antibody or their combination that acts like IL-12 to stimulate IL-12 receptor (e.g., IL-12 or IL-23 mRNA protein/polypeptide/antibody). In some embodiments, the mRNA molecule(s) are injected into the subject directly. In some embodiments, one or more or all of the mRNA molecules are delivered as DNA which are then transcribed into mRNA in vivo.

In some embodiments, the OX40L is a human protein. In some embodiments, the OX40L is a full-length OX40L protein rather than a fragment or domain thereof, such as a soluble portion. In some embodiments, the OX40L is a full-length OX40L protein with different isoforms rather than a fragment or domain thereof, such as a soluble portion.

In some embodiments, the OX40 agonist is a polypeptide that includes at least an extracellular domain of full-length OX40L, which may be fused to a transmembrane domain and optionally an intracellular fragment of another protein.

In some embodiments, the OX40 agonist is a polypeptide that includes the extracellular domain, either alone or fused with a linker fragment (e.g., oligomerization domain) that can promote the formation of its homo-dimers, homo-trimers or homo-oligomers. Protein domains as such as the Fc fragment of immunoglobulins are commonly used to promote formation of homo-dimers.

In some embodiments, the oligomerization domain is capable of formation of homo-trimers (thus a “trimerization domain”). Trimerization domains are known in the art, such as the domains in trimeric proteins responsible for mediating association of the trimeric protein.

Example trimerization domains include the T4 bacteriophage fibritin trimerization motif (T4F), the GCN4 trimeric leucine zipper motif (GCN4), and the human collagen XVIII derived homotrimerization domain (TIE). In some embodiments, the trimerization domain is not longer than 100 amino acids, or not longer than 90, 80, 70, 60, or 50 amino acids.

In some embodiments, the fusion protein further includes a peptide linker between the OX40L extracellular domain and the trimerization domain. In some embodiments, the peptide linker is flexible.

In some embodiments, the distance between an OX40L extracellular domain and the trimerization domain is not longer than 100 amino acids, or not longer than 90, 80, 70, 60, 50, 40, 30, 25, 20, 15 or 10 amino acids. In some embodiments, the peptide linker is from 5 to 50 amino acid residues in length, preferably from 5 to 20 amino acid residues in length.

In some embodiment, the OX40 agonist is an agonist anti-OX40 antibody or an antigen-binding fragment thereof.

In some embodiments, the OX40 agonist mRNA includes the RNA sequence corresponding to the coding sequence of NM_003326 (SEQ ID NO:3). In some embodiments, the OX40L mRNA includes the RNA sequence corresponding to the coding sequence of NM_001297562 (SEQ ID NO:4). In some embodiment, the OX40 agonist mRNA encodes the protein sequence of NP_003317 (SEQ ID NO:1). In some embodiments, the OX40L mRNA encodes the protein sequence of NP_001284491 (SEQ ID NO:2).

In some embodiment, the OX40 agonist mRNA encodes a protein sequence that has at least 70%, 75%, 80%, 85%, 90%, 95%, 98%, or 99% sequence identity to SEQ ID NO:1 or residues 52-183 of SEQ ID NO:1. In some embodiments, the OX40 agonist mRNA encodes a protein sequence that has at least 70%, 75%, 80%, 85%, 90%, 95%, 98%, or 99% sequence identity to SEQ ID NO:2 or residues 2-133 of SEQ ID NO:2. In some embodiments, the protein sequence retains the activity of human OX40L or activates OX40.

In some embodiments, the ( ) 40 agonist mRNA encodes the extracellular domain of NP_003317 (i.e., residues 52-183 of SEQ ID NO:1, or a peptide having at least 70%, 75%, 80%, 85%, 90%, 95%, 98%, or 99% sequence identity to residues 52-183 of SEQ ID NO:1). In some embodiments, the OX40 agonist mRNA encodes the extracellular domain of NP 001284491 (i.e., residues 2-133 of SEQ ID) NO: 2, or a peptide having at least 70%, 75%, 80%, 85%, 90%, 95%, 98%, or 99% sequence identity to residues 2-133 of SEQ ID NO:2).

In some embodiments, the extracellular domain of OX40L can be fused to the transmembrane domain and optionally an intracellular fragment of another protein, such that the fusion protein can be anchored to the plasma membrane. The transmembrane domain and intracellular fragment can be from any protein, such as a human protein, in particularly those that are expressed on the membranes of cells in a tissue where OX40L is desired to be expressed.

A transmembrane domain may be derived either from any membrane-bound or transmembrane protein, such as an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, the IL-12 is human IL-12. In some embodiments, the IL-12 includes IL-12A (p35). In some embodiments, the IL-12 includes IL-12B (p40). In some embodiment, the IL-12 mRNA includes a mRNA encoding IL-12A and a mRNA encoding IL-12B. In some embodiment, the IL-12 mRNA includes a mRNA encoding both IL-12A and IL-12B.

In some embodiments, the IL-12A mRNA includes the mRNA sequence corresponding to the coding sequence of NM_000882 (SEQ ID NO:9). In some embodiments, the IL-12A mRNA encodes the protein sequence of NP_000873 (SEQ ID NO: 5), or the mature protein (residues 57-253 of SEQ ID NO:5).

In some embodiments, the IL-12A mRNA includes the mRNA sequence corresponding to the coding sequence of NM_001354582 (SEQ ID NO:10). In some embodiments, the IL-12A mRNA encodes the protein sequence of NP_001341511 (SEQ ID NO: 6), or the mature protein (residues 57-239 of SEQ ID NO:6).

In some embodiments, the IL-12A mRNA includes the mRNA sequence corresponding to the coding sequence of NM_001354583 (SEQ ID NO:11). In some embodiments, the IL-12A mRNA encodes the protein sequence of NP_001341512 (SEQ ID NO: 7), or the mature protein (residues 57-215 of SEQ ID NO:7).

In some embodiments, the IL-12A mRNA includes the mRNA sequence corresponding to the coding sequence of NM_001397992 (SEQ ID NO:12). In some embodiments, the IL-12A mRNA encodes the protein sequence of NP_001384921 (SEQ ID NO: 8), or the mature protein (residues 23-219 of SEQ ID NO:8).

In some embodiments, the IL-12B mRNA includes the mRNA sequence corresponding to the coding sequence of NM_002187 (SEQ ID NO:14). In some embodiments, the IL-12A mRNA encodes the protein sequence of NP_002178 (SEQ ID NO: 13), or the mature protein (residues 23-328 of SEQ ID NO:13).

TABLE 1
Sequences

| | | SEQ |
|---|---|---|
| Name | Sequence | ID NO: |
| OX40L Isoform 1 | MERVQPLEENVGNAARPRFERNKLLLVASVIQGLGLLLCFTYICLHFSAL | 1 |
| protein | QVSHRYPRIQSIKVQFTEYKKEKGFILTSQKEDEIMKVQNNSVIINCDGF | |
| (NP_003317) | YLISLKGYFSQEVNISLHYQKDEEPLFQLKKVRSVNSLMVASLTYKDKVY | |
| (extracellular: 52- | LNVTTDNTSLDDFHVNGGELILIHQNPGEFCVL | |
| 183 (underlined)) | | |
| OX40L Isoform 2 | MVSHRYPRIQSIKVQFTEYKKEKGFILTSQKEDEIMKVQNNSVIINCDGF | 2 |
| protein | YLISLKGYFSQEVNISLHYQKDEEPLFQLKKVRSVNSLMVASLTYKDKVY | |
| (NP_001284491) | LNVTTDNTSLDDFHVNGGELILIHQNPGEFCVL | |
| OX40L Isoform 1 | auggaaaggguccaaccccuggaagagaaugugggaaaugcagccaggcc | 3 |
| mRNA | aagauucgagaggaacaagcuauugcugguggccucuguaauucagggac | |
| (NM_003326) | uggggcugcuccugugcuucaccuacaucugccugcacuucucugcucuu | |
| coding portion | cagguaucacaucgguauccucgaauucaaaguaucaaaguacaauuuac | |
| | cgaauauaagaaggagaaagguuucauccucacuucccaaaaggaggaug | |
| | aaaucaugaaggugcagaacaacucagucaucaucaacugugauggguuu | |
| | uaucucaucucccugaagggcuacuucucccaggaagucaacauuagccu | |
| | ucauuaccagaaggaugaggagccccucuuccaacugaagaaggucaggu | |
| | cugucaacuccuugaugguggccucucugacuuacaaagacaaagucuac | |
| | uugaaugugaccacugacaauaccucccuggaugacuuccaugugaaugg | |
| | ga | |
| OX40L Isoform 2 | augguaucacaucgguauccucgaauucaaaguaucaaaguacaauuuac | 4 |
| mRNA | cgaauauaagaaggagaaagguuucauccucacuucccaaaaggaggaug | |
| (NM_001297562) | aaaucaugaaggugcagaacaacucagucaucaucaacugugauggguuu | |
| | uaucucaucucccugaagggcuacuucucccaggaagucaacauuagccu | |
| | ucauuaccagaaggaugaggagccccucuuccaacugaagaaggucaggu | |
| | cugucaacuccuugaugguggccucucugacuuacaaagacaaagucuac | |
| | uugaaugugaccacugacaauaccucccuggaugacuuccaugugaaugg | |
| coding portion | cggagaacugauucuuauccaucaaaauccuggugaauucuguguccuuu | |
| | ga | |
| IL-12A isoform 1 | MWPPGSASQPPPSPAAATGLHPAARPVSLQCRLSMCPARSLLLVATLVLL | 5 |
| protein | DHLSLARNLPVATPDPGMFPCLHHSQNLLRAVSNMLQKARQTLEFYPCTS | |
| (NP_000873) | EEIDHEDITKDKTSTVEACLPLELTKNESCLNSRETSFITNGSCLASRKT | |
| (mature protein: | SFMMALCLSSIYEDLKMYQVEFKTMNAKLLMDPKRQIFLDQNMLAVIDEL | |
| 57-253 | MQALNFNSETVPQKSSLEEPDFYKTKIKLCILLHAFRIRAVTIDRVMSYL | |
| (underlined)) | NAS | |
| IL-12A isoform 2 | MWPPGSASQPPPSPAAATGLHPAARPVSLQCRLSMCPARSLLLVATLVLL | 6 |
| protein | DHLSLARNLPVATPDPGMFPCLHHSQNLLRAVSNMLQKARQTLEFYPCTS | |
| (NP_001341511) | EEIDHEDITKDKTSTVEACLPLELTKNGSCLASRKTSFMMALCLSSIYED | |
| (mature protein: | LKMYQVEFKTMNAKLLMDPKRQIFLDQNMLAVIDELMQALNFNSETVPQK | |
| 57-239 | SSLEEPDFYKTKIKLCILLHAFRIRAVTIDRVMSYLNAS | |
| (underlined)) | | |
| IL-12A isoform 3 | MWPPGSASQPPPSPAAATGLHPAARPVSLQCRLSMCPARSLLLVATLVLL | 7 |
| protein | DHLSLARNLPVATPDPGMFPCLHHSQNLLRAVSNMLQKNESCLNSRETSF | |
| (NP_001341512) | ITNGSCLASRKTSFMMALCLSSIYEDLKMYQVEFKTMNAKLLMDPKRQIF | |
| (mature protein: | LDQNMLAVIDELMQALNENSETVPQKSSLEEPDFYKTKIKLCILLHAFRI | |
| 57-215 | RAVTIDRVMSYLNAS | |
| (underlined)) | | |
| IL-12A isoform 4 | MCPARSLLLVATLVLLDHLSLARNLPVATPDPGMFPCLHHSQNLLRAVSN | 8 |
| protein | MLQKARQTLEFYPCTSEEIDHEDITKDKTSTVEACLPLELTKNESCLNSR | |
| (NP_001384921) | ETSFITNGSCLASRKTSFMMALCLSSIYEDLKMYQVEFKTMNAKLLMDPK | |
| (mature protein: | RQIFLDQNMLAVIDELMQALNFNSETVPQKSSLEEPDFYKTKIKLCILLH | |
| 23-219 | AFRIRAVTIDRVMSYLNAS | |
| (underlined)) | | |
| IL-12A isoform 1 | auguggcccccugggucagccucccagccacegcccucaccugcegcggc | 9 |
| mRNA | cacaggucugcauccagcggcucgcccugugucccugcagugccggcuca | |
| (NM_000882) | gcauguguccagcgcgcagccuccuccuuguggcuacccugguccuccug | |
| coding portion | gaccaccucaguuuggccagaaaccuccccguggccacuccagacccagg | |
| | aauguucccaugccuucaccacucccaaaaccugcugagggccgucagca | |
| | acaugcuccagaaggccagacaaacucuagaauuuuacccuugcacuucu | |
| | gaagagauugaucaugaagauaucacaaaagauaaaaccagcacagugga | |
| | ggccuguuuaccauuggaauuaaccaagaaugagaguugccuaaauucca | |
| | gagagaccucuuucauaacuaaugggaguugccuggccuccagaaagacc | |
| | ucuuuuaugauggcccugugccuuaguaguauuuaugaagacuugaagau | |
| | guaccagguggaguucaagaccaugaaugcaaagcuucugauggauccua | |
| | agaggcagaucuuucuagaucaaaacaugcuggcaguuauugaugagcug | |
| | augcaggcccugaauuucaacagugagacugugccacaaaaauccucccu | |
| | ugaagaaccggauuuuuauaaaacuaaaaucaagcucugcauacuucuuc | |
| | augcuuucagaauucgggcagugacuauugauagagugaugagcuaucug | |
| | aaugcuuccuaa | |
| IL-12A isoform 2 | auguggcccccugggucagccucccagccacegcccucaccugccgcggc | 10 |
| mRNA | cacaggucugcauccageggcucgcccugugucccugcagugccggcuca | |
| (NM_001354582) | gcauguguccagcgcgcagccuccuccuuguggcuacccugguccuccug | |
| coding portion | gaccaccucaguuuggccagaaaccuccccguggccacuccagacccagg | |
| | aauguucccaugccuucaccacucccaaaaccugcugagggcegucagca | |
| | acaugcuccagaaggccagacaaacucuagaauuuuacccuugcacuucu | |
| | gaagagauugaucaugaagauaucacaaaagauaaaaccagcacagugga | |
| | ggccuguuuaccauuggaauuaaccaagaaugggaguugccuggccucca | |
| | gaaagaccucuuuuaugauggcccugugccuuaguaguauuuaugaagac | |
| | uugaagauguaccagguggaguucaagaccaugaaugcaaagcuucugau | |
| | ggauccuaagaggcagaucuuucuagaucaaaacaugcuggcaguuauug | |
| | augagcugaugcaggcccugaauuucaacagugagacugugccacaaaaa | |
| | uccucccuugaagaaccggauuuuuauaaaacuaaaaucaagcucugcau | |
| | acuucuucaugcuuucagaauucgggcagugacuauugauagagugauga | |
| | gcuaucugaaugcuuccuaa | |
| IL-12A isoform 3 | auguggcccccugggucagccucccagccaccgcccucaccugccgcggc | 11 |
| mRNA | cacaggucugcauccageggcucgcccugugucccugcagugccggcuca | |
| (NM_001354583) | gcauguguccagcgcgcagccuccuccuuguggcuacccugguccuccug | |
| coding portion | gaccaccucaguuuggccagaaaccuccccguggccacuccagacccagg | |
| | aauguucccaugccuucaccacucccaaaaccugcugagggccgucagca | |
| | acaugcuccagaagaaugagaguugccuaaauuccagagagaccucuuuc | |
| | auaacuaaugggaguugccuggccuccagaaagaccucuuuuaugauggc | |
| | ccugugccuuaguaguauuuaugaagacuugaagauguaccagguggagu | |
| | ucaagaccaugaaugcaaagcuucugauggauccuaagaggcagaucuuu | |
| | cuagaucaaaacaugcuggcaguuauugaugagcugaugcaggcccugaa | |
| | uuucaacagugagacugugccacaaaaauccucccuugaagaaccggauu | |
| | uuuauaaaacuaaaaucaagcucugcauacuucuucaugcuuucagaauu | |
| | cgggcagugacuauugauagagugaugagcuaucugaaugcuuccuaa | |
| IL-12A isoform 4 | auguguccagcgcgcagccuccuccuuguggcuacccugguccuccugga | 12 |
| mRNA | ccaccucaguuuggccagaaaccuccccguggccacuccagacccaggaa | |
| (NM_001397992) | uguucccaugccuucaccacucccaaaaccugcugagggccgucagcaac | |
| coding portion | augcuccagaaggccagacaaacucuagaauuuuacccuugcacuucuga | |
| | agagauugaucaugaagauaucacaaaagauaaaaccagcacaguggagg | |
| | ccuguuuaccauuggaauuaaccaagaaugagaguugccuaaauuccaga | |
| | gagaccucuuucauaacuaaugggaguugccuggccuccagaaagaccuc | |
| | uuuuaugauggcccugugccuuaguaguauuuaugaagacuugaagaugu | |
| | accagguggaguucaagaccaugaaugcaaagcuucugauggauccuaag | |
| | aggcagaucuuucuagaucaaaacaugcuggcaguuauugaugagcugau | |
| | gcaggcccugaauuucaacagugagacugugccacaaaaauccucccuug | |
| | aagaaccggauuuuuauaaaacuaaaaucaagcucugcauacuucuucau | |
| | gcuuucagaauucgggcagugacuauugauagagugaugagcuaucugaa | |
| | ugcuuccuaa | |
| IL-12B protein | MCHQQLVISWFSLVFLASPLVAIWELKKDVYVVELDWYPDAPGEMVVLTC | 13 |
| (NP_002178) | DTPEEDGITWTLDQSSEVLGSGKTLTIQVKEFGDAGQYTCHKGGEVLSHS | |
| (mature protein: | LLLLHKKEDGIWSTDILKDQKEPKNKTFLRCEAKNYSGRFTCWWLTTIST | |
| 23-328 | DLTFSVKSSRGSSDPQGVTCGAATLSAERVRGDNKEYEYSVECQEDSACP | |
| (underlined)) | AAEESLPIEVMVDAVHKLKYENYTSSFFIRDIIKPDPPKNLQLKPLKNSR | |
| | QVEVSWEYPDTWSTPHSYFSLTFCVQVQGKSKREKKDRVFTDKTSATVIC | |
| | RKNASISVRAQDRYYSSSWSEWASVPCS | |
| IL-12B mRNA | aucuccccucguggccauaugggaacugaagaaagauguuuaugucguag | 14 |
| (NM_002187) | aauuggauugguauccggaugccccuggagaaauggugguccucaccugu | |
| coding portion | gacaccccugaagaagaugguaucaccuggaccuuggaccagagcaguga | |
| | ggucuuaggcucuggcaaaacccugaccauccaagucaaagaguuuggag | |
| | augcuggccaguacaccugucacaaaggaggcgagguucuaagccauucg | |
| | cuccugcugcuucacaaaaaggaagauggaauuugguccacugauauuuu | |
| | aaaggaccagaaagaacccaaaaauaagaccuuucuaagaugcgaggcca | |
| | agaauuauucuggacguuucaccugcugguggcugacgacaaucaguacu | |
| | gauuugacauucagugucaaaagcagcagaggcucuucugacccccaagg | |
| | ggugacgugcggagcugcuacacucucugcagagagagucagaggggaca | |
| | acaaggaguaugaguacucaguggagugccaggaggacagugccugccca | |
| | gcugcugaggagagucugcccauugaggucaugguggaugccguucacaa | |
| | gcucaaguaugaaaacuacaccagcagcuucuucaucagggacaucauca | |
| | aaccugacccacccaagaacuugcagcugaagccauuaaagaauucucgg | |
| | cagguggaggucagcugggaguacccugacaccuggaguacuccacauuc | |
| | cuacuucucccugacauucugcguucagguccagggcaagagcaagagag | |
| | aaaagaaagauagagucuucacggacaagaccucagccacggucaucugc | |
| | cgcaaaaaugccagcauuagcguggggcccaggaccgcuacuauagcuc | |
| | aucuuggagegaaugggcaucugugcccugcaguua | |
| GM-CSF protein | MWLQSLLLLGTVACSISAPARSPSPSTQPWEHVNAIQEARRLLNLSRDTA | 15 |
| (NP_000749) | AEMNETVEVISEMFDLQEPTCLQTRLELYKQGLRGSLTKLKGPLTMMASH | |
| (mature protein: | YKQHCPPTPETSCATQIITFESFKENLKDFLLVIPFDCWEPVQE | |
| 18-144 | | |
| (underlined)) | | |
| GM-CSF mRNA | auguggcugcagagccugcugcucuugggcacuguggccugcagcaucuc | 16 |
| (NM_000758) | ugcacccgcccgcucgcccagccccagcacgcagcccugggagcauguga | |
| coding portion | augccauccaggaggcccggcgucuccugaaccugaguagagacacugcu | |
| | gcugagaugaaugaaacaguagaagucaucucagaaauguuugaccucca | |
| | ggagccgaccugccuacagacccgccuggagcuguacaagcagggccugc | |
| | ggggcagccucaccaagcucaagggccccuugaccaugauggccagccac | |
| | uacaagcagcacugcccuccaaccccggaaacuuccugugcaacccagau | |
| | uaucaccuuugaaaguuucaaagagaaccugaaggacuuucugcuuguca | |
| | uccccuuugacugcugggagccaguccaggaguga | |

In some embodiments, the OX40L mRNA (or mRNA encoding a protein/polypeptide/antibody or their combination that activates OX40) and the IL-12 mRNA are separate mRNA molecules. In some embodiments, the OX40L (or mRNA encoding a protein/polypeptide/antibody or their combination that activates OX40) coding sequence and the IL-12 coding sequence are included in the same mRNA molecule which can be translated into a fusion protein/polypeptide or separate proteins/polypeptides.

In some embodiments, one of the mRNA encodes OX40L, such as human OX40L. In some embodiments, the encoded protein has the amino acid sequence of SEQ ID NO:1. In some embodiments, the encoded protein has an amino acid sequence that has at least 70%, 80%, 85%, 90%, 95%, 98%, or 99% sequence identity to SEQ ID NO:1. In some embodiments, a fragment of the encoded protein has an amino acid sequence that has at least 90%, 95%, 98%, or 99% sequence identity to a fragment in SEQ ID NO:1. In some embodiments, the encoded protein has the amino acid sequence of SEQ ID NO:2. In some embodiments, the encoded protein has an amino acid sequence that has at least 70%, 80%, 85%, 90%, 95%, 98%, or 99% sequence identity to SEQ ID NO:2. In some embodiments, a fragment of the encoded protein has an amino acid sequence that has at least 90%, 95%, 98%, or 99% sequence identity to a fragment in SEQ ID NO:2. (Someone may make a fusion protein which contains the extracellular domain of OX40L and the intracellular and transmembrane domains from other proteins).

In some embodiments, the mRNA includes the nucleic acid sequence of SEQ ID NO: 3. In some embodiments, the mRNA includes the nucleic acid sequence that has at least 70%, 80%, 85%, 90%, 95%, 98%, or 99% sequence identity to SEQ ID NO:3. In some embodiments, the mRNA includes the nucleic acid sequence of SEQ ID NO:4. In some embodiments, the mRNA includes the nucleic acid sequence that has at least 70%, 80%, 85%, 90%, 95%, 98%, or 99% sequence identity to SEQ ID NO:4.

In some embodiments, one of the mRNA encodes IL-12A, such as human IL-12A. In some embodiments, the encoded protein has the amino acid sequence of SEQ ID NO:5. In some embodiments, the encoded protein has an amino acid sequence that has at least 70%, 80%, 85%, 90%, 95%, 98%, or 99% sequence identity to SEQ ID NO:5. In some embodiments, the encoded protein has the amino acid sequence of SEQ ID NO:6. In some embodiments, the encoded protein has an amino acid sequence that has at least 70%, 80%, 85%, 90%, 95%, 98%, or 99% sequence identity to SEQ ID NO:6. In some embodiments, the encoded protein has the amino acid sequence of SEQ ID NO:7. In some embodiments, the encoded protein has an amino acid sequence that has at least 70%, 80%, 85%, 90%, 95%, 98%, or 99% sequence identity to SEQ ID NO:7. In some embodiments, the encoded protein has the amino acid sequence of SEQ ID NO:8. In some embodiments, the encoded protein has an amino acid sequence that has at least 70%, 80%, 85%, 90%, 95%, 98%, or 99% sequence identity to SEQ ID NO:8.

In some embodiments, the mRNA includes the nucleic acid sequence of SEQ ID NO: 9. In some embodiments, the mRNA includes the nucleic acid sequence that has at least 70%, 80%, 85%, 90%, 95%, 98%, or 99% sequence identity to SEQ ID NO:9. In some embodiments, the mRNA includes the nucleic acid sequence of SEQ ID NO: 10. In some embodiments, the mRNA includes the nucleic acid sequence that has at least 70%, 80%, 85%, 90%, 95%, 98%, or 99% sequence identity to SEQ ID NO:10. In some embodiments, the mRNA includes the nucleic acid sequence of SEQ ID NO:11. In some embodiments, the mRNA includes the nucleic acid sequence that has at least 70%, 80%, 85%, 90%, 95%, 98%, or 99% sequence identity to SEQ ID NO:11. In some embodiments, the mRNA includes the nucleic acid sequence of SEQ ID NO:12. In some embodiments, the mRNA includes the nucleic acid sequence that has at least 70%, 80%, 85%, 90%, 95%, 98%, or 99% sequence identity to SEQ ID NO: 12.

In some embodiments, one of the mRNA encodes IL-12B, such as human IL-12B. In some embodiments, the encoded protein has the amino acid sequence of SEQ ID NO:13. In some embodiments, the encoded protein has an amino acid sequence that has at least 70%, 80%, 85%, 90%, 95%, 98%, or 99% sequence identity to SEQ ID NO:13.

In some embodiments, the mRNA includes the nucleic acid sequence of SEQ ID NO: 14. In some embodiments, the mRNA includes the nucleic acid sequence that has at least 70%, 80%, 85%, 90%, 95%, 98%, or 99% sequence identity to SEQ ID NO: 14.

In some embodiments, the one or more mRNA encode, collectively OX40L (or a protein polypeptide/antibody or their combination that activates OX40) and IL-12A. In some embodiments, the one or more mRNA encode, collectively OX40L (or a protein polypeptide/antibody or their combination that activates OX40) and IL-12B. In some embodiments, the one or more mRNA encode, collectively OX40L (or a protein polypeptide/antibody or their combination that activates OX40), IL-12A and IL-12B.

When the mRNA molecules encoding IL-12 and OX40L (or a soluble counterpart) are provided on separate molecules, their ratios can be adjusted as needed. In one embodiment, the mass ratio between the IL-12 mRNA and the OX40L mRNA is from 1:0.5 to 1:6, without limitation. In one embodiment, the mass ratio between the IL-12 mRNA and the OX40L mRNA is from 1:0.5 to 1:5. In one embodiment, the mass ratio between the IL-12 mRNA and the OX40L mRNA is from 1:0.5 to 1:4. In one embodiment, the mass ratio between the IL-12 mRNA and the OX40L mRNA is from 1:0.75 to 1:6. In one embodiment, the mass ratio between the IL-12 mRNA and the OX40L mRNA is from 1:0.75 to 1:5. In one embodiment, the mass ratio between the IL-12 mRNA and the OX40L mRNA is from 1:0.75 to 1:4. In one embodiment, the mass ratio between the IL-12 mRNA and the OX40L mRNA is from 1:0.8 to 1:5. In one embodiment, the mass ratio between the IL-12 mRNA and the OX40L mRNA is from 1:0.8 to 1:4. In one embodiment, the mass ratio between the IL-12 mRNA and the OX40L mRNA is from 1:0.8 to 1:3.

In one embodiment, the mass ratio between the IL-12 mRNA and the OX40L mRNA is from 1:0.9 to 1:5. In one embodiment, the mass ratio between the IL-12 mRNA and the OX40L mRNA is from 1:0.9 to 1:4. In one embodiment, the mass ratio between the IL-12 mRNA and the OX40L mRNA is from 1:0.9 to 1:3.5. In one embodiment, the mass ratio between the IL-12 mRNA and the OX40L mRNA is from 1:0.9 to 1:3. In one embodiment, the mass ratio between the IL-12 mRNA and the OX40L mRNA is from 1:0.9 to 1:2.5.

In one embodiment, the mass ratio between the IL-12 mRNA and the OX40L mRNA is from 1:0.9 to 1:2. In one embodiment, the mass ratio between the IL-12 mRNA and the OX40L mRNA is from 1:1 to 1:4. In one embodiment, the mass ratio between the IL-12 mRNA and the OX40L mRNA is from 1:1 to 1:3. In one embodiment, the mass ratio between the IL-12 mRNA and the OX40L mRNA is from 1:1 to 1:2.5. In one embodiment, the mass ratio between the IL-12 mRNA and the OX40L mRNA is from 1:1 to 1:2.

In some embodiments, the OX40L is a full-length OX40L protein. In some embodiments, the OX40L is Fc-OX40L (soluble fragment).

As shown in the experimental examples, addition of other potential therapeutic agents may not increase the therapeutic efficacy of the instant combination of mRNA. Accordingly, in one embodiment, the treatment method or use or composition does not include some other types of mRNA.

In one embodiment, excluded are mRNAs encoding an immune checkpoint inhibitor such as PD-L1, PD-1 and CTLA-4. In one embodiment, excluded are mRNAs encoding an interferon, such as IFN-α, IFN-β, or IFN-γ. In one embodiment, excluded are mRNAs encoding another of the IL-12 family, such as IL-23, IL-27 and IL-35. In one embodiment, excluded are mRNAs encoding other cytokines, such as IL-18.

It is appreciated that one or more of these factors may still be included in certain scenarios. In some embodiments, one or more of these, or one or more others may be included. For instance, in one embodiment, the method, use or composition further includes mRNA encoding part or full length of an immunomodulatory factor, such as CD27, CD28, CD40, CD122, CD137, GITR, GSDMD, A2AR, CD276, VTCN1, BTLA, CTLA-4, IDO, LAG3, KIR, NOX2, PD-1, TIM-3, VISTA, SIGLEC7, SIGLEC9, IL-2, IL15, IL6, IL18, IL23, IFN-α, TNF-β, IFN-γ, GM-CSF, M-CSF, RIG-I, MDA5, cGAS, Toll-like receptors, MAVS/VISA, STING/MITA, TRIF, TBK1, IRF3, IRF7, IRF1, JAK1, JAK2, Tyk2, STAT1, STAT2, STAT3, TNFR and any combination thereof.

In a particular embodiment, the added agent is GM-CSF or a mRNA encoding GM-CSF. In a particular embodiment, the added agent is TNFR or a mRNA encoding TNFR. In a particular embodiment, the added agent is GSDMD or a mRNA encoding GSDMD. For instance, in one embodiment, the method, use or composition further includes small molecule reagents, recombinant proteins, antibodies. In some embodiments, the method further comprises administering to the patient a fourth mRNA encoding TNFR (tumor necrosis factor receptor). In some embodiments, the method further comprises administering to the patient a fourth mRNA encoding GSDMD (Gasdermin D).

In some embodiments, the GM-CSF includes amino acid residues 18-144 of SEQ ID NO: 15, or a sequencing having at least 70%, 75%, 80%, 85%, 90%, 95%, 97%, 98%, or 99% sequence identity to amino acid residues 18-144 of SEQ ID NO:15. In some embodiments, the GM-CSF includes SEQ ID NO: 15, or a sequencing having at least 70%, 75%, 80%, 85%, 90%, 95%, 97%, 98%, or 99% sequence identity to SEQ ID NO: 15. In some embodiment, the mRNA encoding GM-CSF includes SEQ ID NO:16, or has at least 70%, 75%, 80%, 85%, 90%, 95%, 97%, 98%, or 99% sequence identity to SEQ ID NO:16.

mRNAs may be synthesized according to any of a variety of known methods. For example, the mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, SP6 or other RNA polymerase), DNAse I, pyrophosphatase, and/or RNAse inhibitor. The exact conditions will vary according to the specific application.

In some embodiments, for the preparation of mRNA, a DNA template is transcribed in vitro. A suitable DNA template typically has a promoter, for example a T3, T7, SP6 or other RNA polymerase promoter, for in vitro transcription, followed by desired nucleotide sequence for desired mRNA and a termination signal.

Desired mRNA sequence may be determined and incorporated into a DNA template using standard methods. For example, starting from a desired amino acid sequence, a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

The mRNA includes linear RNA, circular RNA and any other form of RNA. The mRNA may be synthesized as unmodified or modified mRNA. In some embodiments, the mRNA is modified to enhance stability. In some embodiments, the mRNA is modified to reduce immunogenicity. In some embodiments, the mRNA is modified to enhance efficiency of translation.

It is contemplated that for certain application of the present technology, the mRNA used are not modified to reduce immunogenicity, which is beneficial to the treatment efficacy. In some embodiments, each mRNA does not include chemical modification that reduces immunogenicity. In some embodiments, each mRNA does not include chemical modification to the backbone. In some embodiments, each mRNA only includes natural nucleosides.

Modifications of mRNA can include, for example, modifications of the nucleotides of the RNA. A modified mRNA can thus include, for example, backbone modifications, sugar modifications or base modifications. In some embodiments, the mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 13-D-mannosyl-queosine, wybutoxosine, and phosphoramidates, phosphorothioates, peptide nucleotides, methylphosphonates, 7-deazaguanosine, 5-methylcytosine and inosine. In some embodiments, at least one of the uridine nucleosides in the mRNAs are chemically modified. In some embodiments, the chemically modified uridine nucleosides are N1-methylpseudouridines.

In some embodiments, the mRNAs may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, the mRNAs may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, the mRNAs may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

In some embodiments, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, the mRNAs include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G (5′) ppp (5′(A,G(5′)ppp(5)A and G(5)ppp(5′)G.

In some embodiments, the mRNAs include a 3′ poly(A) tail structure. A poly-A tail on the 3′ terminus of mRNA typically includes about 10 to 300 adenosine nucleotides (e.g., about 10 to 200 adenosine nucleotides, about 10 to 175 adenosine nucleotides, about 10 to 150 adenosine nucleotides, about 10 to 125 adenosine nucleotides, 10 to 100 adenosine nucleotides, about 10 to 75 adenosine nucleotides, about 20 to 70 adenosine nucleotides, or about 20 to 60 adenosine nucleotides). In some embodiments, the include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, the mRNAs include a 5′ and or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length (e.g., about 50 and 400 nucleotides in length, about 50 and 300 nucleotides in length, about 50 and 200 nucleotides in length, or about 50 and 100 nucleotides in length).

In some embodiments, a 5′ region of an mRNA includes a sequence encoding a signal peptide, such as those described herein. Typically, a signal peptide encoding sequence is linked, directly or indirectly, to the coding sequence at the N-terminus.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

In some embodiments, the mRNA is packaged with a delivery agent. In some embodiments, the delivery agent includes a lipidoid, a liposome, a lipoplex, a lipid nanoparticle (LNP), a polymeric compound, a peptide, a protein, a cell, a nanoparticle mimic, a nanotube, a conjugate, or any other delivery material. In some embodiments, the delivery agent is a lipid nanoparticle. In some embodiments, the lipid nanoparticle includes lipids selected from the group consisting of DLin-DMA, DLin-K-DMA, 98N12-5, C12-200, DLin-MC3-DMA, DLin-KC2-DMA, DODMA, PLGA, PEG, PEG-DMG, PEGylated lipids, amino alcohol lipids, KL22, and combinations thereof.

In one embodiment, the LNP includes a molar ratio of 40-60% ionizable amino lipid, a molar ratio of 8-16% phospholipid, a molar ratio of 30-45% sterol, and a molar ratio of 1-5% PEG-modified lipid. In one embodiment, the LNP includes a molar ratio of 45-65% ionizable amino 40 lipid, a molar ratio of 5-10% phospholipid, a molar ratio of 25-40% sterol, and a molar ratio of 0.5-5% PEG modified lipid. In one embodiment, the LNP includes a molar ratio of 40-60% ionizable amino lipid, a molar ratio of 8-16% phospholipid, a molar ratio of 30-45% sterol, and a molar ratio of 1-5% PEG modified lipid. In one embodiment, the LNP includes a molar ratio of 45-65% ionizable amino lipid, a molar ratio of 5-10% phospholipid, a molar ratio of 25-40% sterol, and a molar ratio of 0.5-5% PEG modified lipid. In one embodiment, the LNP includes a molar ratio of 40-60% ionizable amino lipid, a molar ratio of 8-16% phospholipid, a molar ratio of 30-45% sterol, and a molar ratio of 1-5% PEG modified lipid. In one embodiment, the LNP includes a molar ratio of 45-65% ionizable amino lipid, a molar ratio of 5-10% phospholipid, a molar ratio of 25-40% sterol, and a molar ratio of 0.5-5% PEG modified lipid.

In some embodiments, the mRNA is packaged in liposomes. Liposomes can be prepared by various techniques known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then added to the vessel with a vortexing motion which results in the formation of MLVs. Uni-lamellar vesicles (ULV) can then be formed by homogenization, sonication or extrusion of the multi-lamellar vesicles. In addition, unilamellar vesicles can be formed by detergent removal techniques.

In certain embodiments, the mRNA is associated on both the surface of the liposome and encapsulated within the same liposome. For example, during preparation of the compositions of the present invention, cationic liposomes may associate with the mRNA through electrostatic interactions.

In some embodiments, the mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (Zeta potential), targeting ligands and/or combinations thereof. In some embodiments, the one or more liposome may have a different composition of cationic lipids, neutral lipid, PEG-modified lipid and/or combinations thereof. In some embodiments the one or more liposomes may have a different molar ratio of cationic lipid, neutral lipid, cholesterol and PEG-modified lipid used to create the liposome.

In some embodiments, the liposome includes a cationic lipid, a non-cationic lipid, a cholesterol-based lipid and a PEG modified lipid. In some embodiments, the cationic lipid is selected from the group consisting of 1,1′-((2-(4-(2-((2-(bis(2-hydroxydodecyl)amino)ethyl) 2-hydroxydodecyl)amino)ethyl)piperazin-1-yl)ethyl)azanediyl)bis(dodecan-2-ol) (C12-200), (6Z,9Z,28Z,31Z)-heptatriaconta-6,9,28,31-tetraen-19-yl 4-(dimethylamino)butanoate (MC3), N,N-dimethyl-2,3-bis((9Z,12Z)-octadeca-9,12-dien-1-yloxy) propan-1-amine (DLinDMA), 2-(2,2-di((9Z,12Z)-octadeca-9,12-dien-1-yl)-1,3-dioxolan-4-yl)-N,N-dimethylethanamine (DLinKC2DMA, [XTC2]), 3,6-bis(4-(bis(2-hydroxydodecyl)amino)butyl)piperazine-2,5-dione (cKK-E12), 10,13-dimethyl-17-(6-methylheptan-2-yl)-2,3,4,7,8,9,10,11,12,13,14,15,16,17-tetradecahydro-1H-cyclopenta[a]phenanthren-3-yl 3-(1H-imidazol-5-yl) propanoate (ICE), (15Z,18Z)-N,N-dimethyl-6-((9Z,12Z)-octadeca-9,12-dien-1-yl)tetracosa-15,18-dien-1-amine (HGT5000), (4Z,15Z,18Z)-N,N-dimethyl-6-((9Z,12Z)-octadcca-9,12-dien-1-yl)tetracosa-4,15,18-trien-1-amine (HGT5001), N,N-diolcyl-N,N-dimethylammonium chloride (DODAC), N,N-distearyl-N,N-dimethylammonium bromide (DDAB), 1,2-dimyristyloxyproyl-3-dimethyl-hydroxyethyl ammonium bromide (DMRIE), dioleoyloxy-N-[2-sperminecarboxamido)ethyl]-N,N-dimethyl-1-propaniminiumtrifluoroacetate (DOSPA), dioctadecylamidoglycyl spermine (DOGS), 1,2-dioleoyl-3-dimethylammonium-propane (DODAP), N,N-dimethyl-(2,3-dioleyloxy) propylamine (DODMA) and N,N-dimethyl-(2,3-dimyristyloxy) propylamine (DMDMA), 1,2-dilinolenyloxy-N,N-dimethylaminopropane (DLenDMA), (2S)-2-(4-((10,13-dimethyl-17-(6-methylheptan-2-yl)-2,3,4,7,8,9,10,11,12,13, 14, 15, 16, 17-tetradecahydro-1H-cyclopenta[a]phenanthren-3-yl)oxy) butoxy)-N,N-dimethyl-3-((9Z,12Z)-octadeca-9,12-dien-1-yloxy) propan-1-amine (CLinDMA), 2-[5′-(cholest-5-en-3 [betal]-oxy)-3′-oxapentoxy)-3-dimethyl-1-1 (cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), (9Z,9′Z,12Z,12′Z)-3-(dimethylamino) propane-1,2-diyl bis(octadeca-9,12-dienoate) (DLinDAP), 1,2-dilinolcylcarbamyl-3-dimethylaminopropane (DLinCDAP), 2,2-dilinoleyl-4-dimethylaminomethyl-[1,3]-dioxolane (DLin-K-DMA), 2-((2,3-bis((9Z,12Z)-octadeca-9,12-dien-1-yloxy) propyl)disulfanyl)-N,N-dimethylethanamine (HGT4003), and combinations thereof.

In some embodiments, the cholesterol-based lipid is cholesterol or PEGylated cholesterol. In some embodiments, the cationic lipid constitutes about 30-50% of the liposome by molar ratio. In some embodiments, the ratio of cationic lipid:non-cationic lipid:cholesterollipid:PEGylated lipid is approximately 50:10:35:5 by molar ratio.

In some embodiments, the liposome includes a combination selected from the group consisting of: cKK-E12, 1,2-dioleyl-sn-glycero-3-phosphoethanolamine (DOPE), cholesterol and 1,2-dimyristoyl-sn-glycerol, methoxypolyethylene Glycol (DMG-PEG2K); C12-200, DOPE, cholesterol and DMG-PEG2K; HGT4003, DOPE, cholesterol and DMG-PEG2K; or ICE, DOPE, cholesterol and DMG-PEG2K.

Suitable liposomes may be made in various sizes. In some embodiments, provided liposomes may be made smaller than previously known mRNA encapsulating liposomes. In some embodiments, decreased size of liposomes is associated with more efficient delivery of mRNA. Selection of an appropriate liposome size may take into consideration the site of the target cell or tissue and to some extent the application for which the liposome is being made.

In some embodiments, a suitable liposome has a size no greater than about 250 nm (e.g., no greater than about 225 nm, 200 nm, 175 nm, 150 nm, 125 nm, 100 nm, 75 nm, or 50 nm). In some embodiments, a suitable liposome has a size ranging from about 10-250 nm (e.g., ranging from about 10-225 nm, 10-200 nm, 10-175 nm, 10-150 nm, 10-125 nm, 10-100 nm, 10-75 nm, or 10-50 nm). In some embodiments, a suitable liposome has a size ranging from about 100-250 nm (e.g., ranging from about 100-225 nm, 100-200 nm, 100-175 nm, 100-150 nm). In some embodiments, a suitable liposome has a size ranging from about 10-100 nm (e.g., ranging from about 10-90 nm, 10-80 nm, 10-70 nm, 10-60 nm, or 10-5 nm).

According to various embodiments, the timing of expression of delivered mRNAs can be tuned to suit a particular medical need. In some embodiments, the expression of the OX40L protein encoded by delivered mRNA is detectable 1, 2, 3, 6, 12, 18, 24, 30, 36, 42, 48, 54, 60, 66, and/or 72 hours in serum or target tissues after a single administration of provided liposomes or compositions. In some embodiments, the expression of the OX40L protein encoded by the mRNA is detectable 1 day, 2 days, 3 days, 4 days, 5 days, 6 days, and/or 7 days in serum or target tissues after a single administration of provided liposomes or compositions. In some embodiments, the expression of the OX40L protein encoded by the mRNA is detectable 1 week, 2 weeks, 3 weeks, and/or 4 weeks in serum or target tissues after a single administration of provided liposomes or compositions. In some embodiments, the expression of the protein encoded by the mRNA is detectable after a month or longer after a single administration of provided liposomes or compositions.

In some embodiments, the expression of the IL-12A protein encoded by delivered mRNA is detectable 1, 2, 3, 6, 12, 18, 24, 30, 36, 42, 48, 54, 60, 66, and/or 72 hours in serum or target tissues after a single administration of provided liposomes or compositions. In some embodiments, the expression of the IL12-A protein encoded by the mRNA is detectable 1 day, 2 days, 3 days, 4 days, 5 days, 6 days, and/or 7 days in serum or target tissues after a single administration of provided liposomes or compositions. In some embodiments, the expression of the IL-12A protein encoded by the mRNA is detectable 1 week, 2 weeks, 3 weeks, and/or 4 weeks in serum or target tissues after a single administration of provided liposomes or compositions. In some embodiments, the expression of the protein encoded by the mRNA is detectable after a month or longer after a single administration of provided liposomes or compositions. In some embodiments, the expression of the IL-12B protein encoded by delivered mRNA is detectable 1, 2, 3, 6, 12, 18, 24, 30, 36, 42, 48, 54, 60, 66, and/or 72 hours in serum or target tissues after a single administration of provided liposomes or compositions. In some embodiments, the expression of the IL-12B protein encoded by the mRNA is detectable 1 day, 2 days, 3 days, 4 days, 5 days, 6 days, and/or 7 days in scrum or target tissues after a single administration of provided liposomes or compositions. In some embodiments, the expression of the IL-12B protein encoded by the mRNA is detectable 1 week, 2 weeks, 3 weeks, and/or 4 weeks in serum or target tissues after a single administration of provided liposomes or compositions. In some embodiments, the expression of the protein encoded by the mRNA is detectable after a month or longer after a single administration of provided liposomes or compositions.

A specific dosage and treatment regimen for any particular patient will depend upon a variety of factors, including the particular mRNA, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the mRNA used, the severity of the disease, and the desired effect. The amount used can be determined by pharmacological and pharmacokinetic principles well known in the art.

Methods of administration of the mRNA include but are not limited to intradermal, intramuscular, intraperitoneal, intravenous, subcutaneous, intranasal, and epidural. The term “parenteral” as used herein refers to modes of administration which include intravenous, intramuscular, intraperitoneal, intrasternal, subcutaneous and intra-articular injection and infusion. In some embodiments, the administration is intratumoral injection. In some embodiments, the administration is subcutaneous injection. In some embodiments, the administration is intramuscular or intravenous injection. In some embodiments, the administration is subcutaneous injection, intramuscular injection, intraperitoneal injection, thoracic injection, intravenous injection, arterial injection, or a combination thereof.

In some embodiments, the injection is into a tumor tissue. In some embodiments, the injection is into one side, such as an end or a portion, of a tumor tissue. In some embodiments, the injection is not into a tumor tissue.

In some embodiments, the administration is made at a frequency of 3 times a week, twice a weekly, once a week, once every 2 weeks, once every 3 weeks, once every 4 weeks, once a month, or once every 3-6 months.

Cancers that can be suitably treated with the present technology include solid tumors, leukemia and lymphoma. In some embodiments, the cancer is squamous cell carcinoma, lung cancer, peritoneal cancer, hepatocellular carcinoma, gastric cancer, pancreatic cancer, glioblastoma, cervical cancer, ovarian cancer, liver cancer, bladder cancer, urethral cancer, breast cancer, colon cancer, rectal cancer, colorectal cancer, endometrial cancer, uterine cancer, salivary gland cancer, kidney cancer, prostate cancer, vulvar cancer, thyroid cancer, liver cancer, anal cancer, soft tissue sarcoma, neuroblastoma, penile cancer, melanoma, superficial spreading melanoma, lentigines melanoma, acral melanoma, nodular melanoma, multiple bone marrow Tumor, B-cell lymphoma, chronic lymphocytic leukemia, non-Hodgkin's lymphoma, acute lymphoblastic leukemia, hairy cell leukemia, chronic myeloblastic leukemia, post-transplant lymphoproliferative disorders, brain tumors, brain cancer and head and neck cancer.

In one embodiment, the cancer is a solid tumor. In one embodiment, the cancer is metastatic. In one embodiment, the cancer is colon cancer. In one embodiment, the cancer is breast cancer, including triple negative breast cancer. In one embodiment, the cancer is lung cancer.

Direct Protein Delivery or Hybrid with mRNA

In some of the above embodiments, both (or more) agents (e.g., IL-12 and OX40L) are delivered as encoding mRNA molecule(s). In alternative embodiments, one or more of the agents can be delivered directly as proteins. As demonstrated in Example 9, for instance, whether the agonist anti-OX40 antibody was delivered as a protein or mRNA, comparable results were achieved. Likewise, the IL-12 protein can be delivered as a protein as well.

According to one embodiment of the present disclosure, therefore, provided is a method for treating cancer that entails administration of a mRNA encoding an OX40 agonist (e.g., OX40L or a protein/polypeptide/antibody or their combination that activates OX40 (e.g., an OX40L protein or anti-OX40 agonist antibody)), and an IL-12 or a protein polypeptide/antibody or their combination that acts like IL-12 to stimulate IL-12 receptor (e.g., IL-12 or IL-23 mRNA/protein/polypeptide/antibody).

In another embodiment, provided is a method for treating cancer that entails administration of an OX40 agonist (e.g., OX40L or a protein/polypeptide/antibody or their combination that activates OX40 (e.g., an OX40L protein or anti-OX40 agonist antibody)), and an mRNA encoding IL-12 or a protein/polypeptide/antibody or their combination that acts like IL-12 to stimulate IL-12 receptor (e.g., IL-12 or IL-23 mRNA/protein/polypeptide/antibody).

In another embodiment, provided is a method for treating cancer that entails administration of an OX40 agonist (e.g., OX40L or a protein/polypeptide/antibody or their combination that activates OX40 (e.g., an OX40L protein or anti-OX40 agonist antibody)), and an IL-12 or a protein/polypeptide/antibody or their combination that acts like IL-12 to stimulate IL-12 receptor (e.g., IL-12 or IL-23 protein/polypeptide/antibody).

In some embodiments, the OX40L is a human protein. In some embodiments, the OX40L is a full OX40L protein rather than a fragment or domain thereof, such as a soluble portion. In some embodiments, the OX40L is a full OX40L protein with different isoforms rather than a fragment or domain thereof, such as a soluble portion.

In some embodiments, the OX40 agonist is a polypeptide that includes at least an extracellular domain of full-length OX40L, which may be fused to a transmembrane domain and optionally an intracellular fragment of another protein.

In some embodiments, the OX40 agonist is a polypeptide that includes the extracellular domain, either alone or fused with a linker fragment (e.g., oligomerization domain) that can promote the formation of its homo-dimers, homo-trimers or homo-oligomers. Protein domains as such as the Fc fragment of immunoglobulins are commonly used to promote formation of homo-dimers.

Example trimerization domains include the T4 bacteriophage fibritin trimerization motif (T4F), the GCN4 trimeric leucine zipper motif (GCN4), and the human collagen XVIII derived homotrimerization domain (TIE). In some embodiments, the trimerization domain is not longer than 100 amino acids, or not longer than 90, 80, 70, 60, or 50 amino acids.

In some embodiments, the fusion protein further includes a peptide linker between the OX40L extracellular domain and the trimerization domain. In some embodiments, the peptide linker is flexible.

In some embodiments, the distance between an OX40L extracellular domain and the trimerization domain is not longer than 100 amino acids, or not longer than 90, 80, 70, 60, 50, 40, 30, 25, 20, 15 or 10 amino acids. In some embodiments, the peptide linker is from 5 to 50 amino acid residues in length, preferably from 5 to 20 amino acid residues in length.

In some embodiment, the OX40 agonist is an agonist anti-OX40 antibody or an antigen-binding fragment thereof.

In some embodiments, the extracellular domain of OX40L can be fused to the transmembrane domain and optionally an intracellular fragment of another protein, such that the fusion protein can be anchored to the plasma membrane. The transmembrane domain and intracellular fragment can be from any protein, such as a human protein, in particularly those that are expressed on the membranes of cells in a tissue where OX40L is desired to be expressed.

A transmembrane domain may be derived either from any membrane-bound or transmembrane protein, such as an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1. SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, the IL-12 is human IL-12. In some embodiments, the IL-12 includes IL-12A (p35). In some embodiments, the IL-12 includes IL-12B (p40).

In some embodiments, the OX40 agonist is OX40L, such as human OX40L. In some embodiments, the OX40L protein has the amino acid sequence of SEQ ID NO:1. In some embodiments, the OX40L protein has an amino acid sequence that has at least 70%, 80%, 85%, 90%, 95%, 98%, or 99% sequence identity to SEQ ID NO:1. In some embodiments, a fragment of the OX40L protein has an amino acid sequence that has at least 90%, 95%, 98%, or 99% sequence identity to a fragment in SEQ ID NO:1. In some embodiments, the OX40L protein has the amino acid sequence of SEQ ID NO:2. In some embodiments, the OX40L protein has an amino acid sequence that has at least 70%, 80%, 85%, 90%, 95%, 98%, or 99% sequence identity to SEQ ID NO:2. In some embodiments, a fragment of the OX40L protein has an amino acid sequence that has at least 90%, 95%, 98%, or 99% sequence identity to a fragment in SEQ ID NO:2. (Someone may make a fusion protein which contains the extracellular domain of OX40L and the intracellular and transmembrane domains from other proteins).

In some embodiments, the IL-12 includes IL-12A, such as human IL-12A. In some embodiments, the IL-12A protein has the amino acid sequence of SEQ ID NO:5. In some embodiments, the IL-12A protein has an amino acid sequence that has at least 70%, 80%, 85%, 90%, 95%, 98%, or 99% sequence identity to SEQ ID NO:5. In some embodiments, the IL-12A protein has the amino acid sequence of SEQ ID NO:6. In some embodiments, the IL-12A protein has an amino acid sequence that has at least 70%, 80%, 85%, 90%, 95%, 98%, or 99% sequence identity to SEQ ID NO:6. In some embodiments, the IL-12A protein has the amino acid sequence of SEQ ID NO:7. In some embodiments, the IL-12A protein has an amino acid sequence that has at least 70%, 80%, 85%, 90%, 95%, 98%, or 99% sequence identity to SEQ ID NO:7. In some embodiments, the IL-12A protein has the amino acid sequence of SEQ ID NO:8. In some embodiments, the IL-12A protein has an amino acid sequence that has at least 70%, 80%, 85%, 90%, 95%, 98%, or 99% sequence identity to SEQ ID NO: 8.

In some embodiments, the IL-12 protein includes IL-12B, such as human IL-12B. In some embodiments, the IL-12B protein has the amino acid sequence of SEQ ID NO:13. In some embodiments, the IL-12B protein has an amino acid sequence that has at least 70%, 80%, 85%, 90%, 95%, 98%, or 99% sequence identity to SEQ ID NO:13.

In some embodiments, the OX40L is a full-length OX40L protein. In some embodiments, the OX40L is Fc-OX40L (soluble fragment).

As shown in the experimental examples, addition of other potential therapeutic agents may not increase the therapeutic efficacy of the instant combinations. Accordingly, in one embodiment, the treatment method or use or composition does not include some other types of agents.

In one embodiment, excluded are immune checkpoint inhibitors such as PD-L1, PD-1 and CTLA-4. In one embodiment, excluded are interferons, such as IFN-α, IFN-β, or IFN-γ. In one embodiment, excluded are proteins of the IL-12 family, such as IL-23, IL-27 and IL-35. In one embodiment, excluded are other cytokines, such as IL-18.

It is appreciated that one or more of these factors may still be included in certain scenarios. In some embodiments, one or more of these, or one or more others may be included. For instance, in one embodiment, the method, use or composition further includes an immunomodulatory factor, such as CD27, CD28, CD40, CD122, CD137, GITR, GSDMD, A2AR, CD276, VTCN1, BTLA, CTLA-4, IDO, LAG3, KIR, NOX2, PD-1, TIM-3, VISTA, SIGLEC7, SIGLEC9, IL-2, IL15, IL6, IL18, IL23, IFN-α, TNF-β, IFN-γ, GM-CSF, M-CSF, RIG-I, MDA5, cGAS, Toll-like receptors, MAVS/VISA, STING/MITA, TRIF, TBK1, IRF3, IRF7, IRF1, JAK1, JAK2, Tyk2, STAT1, STAT2, STAT3, TNFR and any combination thereof. In a particular embodiment, the added agent is GM-CSF or a mRNA encoding GM-CSF. In a particular embodiment, the added agent is TNFR or a mRNA encoding TNFR. In a particular embodiment, the added agent is GSDMD or a mRNA encoding GSDMD. For instance, in one embodiment, the method, use or composition further includes small molecule reagents, recombinant proteins, antibodies.

A specific dosage and treatment regimen for any particular patient will depend upon a variety of factors, including the particular protein, mRNA, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the protein/mRNA used, the severity of the disease, and the desired effect. The amount used can be determined by pharmacological and pharmacokinetic principles well known in the art.

Methods of administration of the protein/mRNA include but are not limited to intradermal, intramuscular, intraperitoneal, intravenous, subcutaneous, intranasal, and epidural. The term “parenteral” as used herein refers to modes of administration which include intravenous, intramuscular, intraperitoneal, intrasternal, subcutaneous and intra-articular injection and infusion. In some embodiments, the administration is intratumoral injection. In some embodiments, the administration is subcutaneous injection. In some embodiments, the administration is intramuscular or intravenous injection. In some embodiments, the administration is subcutaneous injection, intramuscular injection, intraperitoneal injection, thoracic injection, intravenous injection, arterial injection, or a combination thereof.

In some embodiments, the injection is into a tumor tissue. In some embodiments, the injection is into one side, such as an end or a portion, of a tumor tissue. In some embodiments, the injection is not into a tumor tissue.

In some embodiments, the administration is made at a frequency of 3 times a week, twice a weekly, once a week, once every 2 weeks, once every 3 weeks, once every 4 weeks, once a month, or once every 3-6 months.

Cancers that can be suitably treated with the present technology include solid tumors, leukemia and lymphoma. In some embodiments, the cancer is squamous cell carcinoma, lung cancer, peritoneal cancer, hepatocellular carcinoma, gastric cancer, pancreatic cancer, glioblastoma, cervical cancer, ovarian cancer, liver cancer, bladder cancer, urethral cancer, breast cancer, colon cancer, rectal cancer, colorectal cancer, endometrial cancer, uterine cancer, salivary gland cancer, kidney cancer, prostate cancer, vulvar cancer, thyroid cancer, liver cancer, anal cancer, soft tissue sarcoma, neuroblastoma, penile cancer, melanoma, superficial spreading melanoma, lentigines melanoma, acral melanoma, nodular melanoma, multiple bone marrow Tumor, B-cell lymphoma, chronic lymphocytic leukemia, non-Hodgkin's lymphoma, acute lymphoblastic leukemia, hairy cell leukemia, chronic myeloblastic leukemia, post-transplant lymphoproliferative disorders, brain tumors, brain cancer and head and neck cancer.

In one embodiment, the cancer is a solid tumor. In one embodiment, the cancer is metastatic. In one embodiment, the cancer is colon cancer. In one embodiment, the cancer is breast cancer, including triple negative breast cancer. In one embodiment, the cancer is lung cancer.

### Compositions and Combinations

Combinations, packages, kits and compositions are also provided that are useful for carrying out the methods of the instant disclosure.

One embodiment provides a combination, package, kit, or composition that includes a mRNA encoding an OX40 agonist (e.g., OX40L or a protein/polypeptide/antibody or their combination that activates OX40 (e.g., an OX40L protein or anti-OX40 agonist antibody)), and an mRNA encoding IL-12 or a protein/polypeptide/antibody or their combination that acts like IL-12 to stimulate IL-12 receptor (e.g., IL-12 or IL-23 mRNA/protein/polypeptide/antibody). In some embodiments, the mRNA molecule(s) are injected into the subject directly. In some embodiments, one or more or all of the mRNA molecules are delivered as DNA which are then transcribed into mRNA in vivo.

Another embodiment provides a combination, package, kit, or composition that includes a mRNA encoding an OX40 agonist (e.g., OX40L or a protein/polypeptide/antibody or their combination that activates OX40 (e.g., an OX40L protein or anti-OX40 agonist antibody)), and an IL-12 or a protein/polypeptide/antibody or their combination that acts like IL-12 to stimulate IL-12 receptor (e.g., IL-12 or IL-23 mRNA/protein/polypeptide/antibody).

Another embodiment provides a combination, package, kit, or composition that includes an OX40 agonist (e.g., OX40L or a protein/polypeptide/antibody or their combination that activates OX40 (e.g., an OX40L protein or anti-OX40 agonist antibody)), and an mRNA encoding IL-12 or a protein/polypeptide/antibody or their combination that acts like IL-12 to stimulate IL-12 receptor (e.g., IL-12 or IL-23 mRNA/protein/polypeptide/antibody).

Another embodiment provides a combination, package, kit, or composition that includes an OX40 agonist (e.g., OX40L or a protein/polypeptide/antibody or their combination that activates OX40 (e.g., an OX40L protein or anti-OX40 agonist antibody)), and an IL-12 or a protein/polypeptide/antibody or their combination that acts like IL-12 to stimulate IL-12 receptor (e.g., IL-12 or IL-23 protein/polypeptide/antibody).

In any of the above embodiments, combination, package, kit, or composition further includes a GM-CSF protein, an mRNA encoding GM-CSF, or a DNA construct encoding GM-CSF. In any of the above embodiments, combination, package, kit, or composition further includes a TNFR protein, an mRNA encoding TNFR, or a DNA construct encoding TNFR. In any of the above embodiments, combination, package, kit, or composition further includes a GSDMD protein, an mRNA encoding GSDMD, or a DNA construct encoding GSDMD.

In some embodiments, the OX40L is a human protein. In some embodiments, the OX40L is a full OX40L protein rather than a fragment or domain thereof, such as a soluble portion. In some embodiments, the OX40L is a full OX40L protein with different isoforms rather than a fragment or domain thereof, such as a soluble portion.

In some embodiments, the OX40 agonist is a polypeptide that includes at least an extracellular domain of full-length OX40L, which may be fused to a transmembrane domain and optionally an intracellular fragment of another protein.

In some embodiments, the OX40 agonist is a polypeptide that includes the extracellular domain, either alone or fused with a linker fragment (e.g., oligomerization domain) that can promote the formation of its homo-dimers, homo-trimers or homo-oligomers. Protein domains as such as the Fc fragment of immunoglobulins are commonly used to promote formation of homo-dimers.

Example trimerization domains include the T4 bacteriophage fibritin trimerization motif (T4F), the GCN4 trimeric leucine zipper motif (GCN4), and the human collagen XVIII derived homotrimerization domain (TIE). In some embodiments, the trimerization domain is not longer than 100 amino acids, or not longer than 90, 80, 70, 60, or 50 amino acids.

In some embodiments, the fusion protein further includes a peptide linker between the OX40L extracellular domain and the trimerization domain. In some embodiments, the peptide linker is flexible.

In some embodiments, the distance between an OX40L extracellular domain and the trimerization domain is not longer than 100 amino acids, or not longer than 90, 80, 70, 60, 50, 40, 30, 25, 20, 15 or 10 amino acids. In some embodiments, the peptide linker is from 5 to 50 amino acid residues in length, preferably from 5 to 20 amino acid residues in length.

In some embodiment, the OX40 agonist is an agonist anti-OX40 antibody or an antigen-binding fragment thereof.

In some embodiments, the OX40 agonist mRNA includes the RNA sequence corresponding to the coding sequence of NM_003326 (SEQ ID NO:3). In some embodiments, the OX40L mRNA includes the RNA sequence corresponding to the coding sequence of NM_001297562 (SEQ ID NO:4). In some embodiment, the OX40 agonist mRNA encodes the protein sequence of NP_003317 (SEQ ID NO:1). In some embodiments, the OX40L mRNA encodes the protein sequence of NP_001284491 (SEQ ID NO:2).

In some embodiment, the OX40 agonist mRNA encodes a protein sequence that has at least 70%, 75%, 80%, 85%, 90%, 95%, 98%, or 99% sequence identity to SEQ ID NO:1 or residues 52-183 of SEQ ID NO:1. In some embodiments, the OX40 agonist mRNA encodes a protein sequence that has at least 70%, 75%, 80%, 85%, 90%, 95%, 98%, or 99% sequence identity to SEQ ID NO:2 or residues 2-133 of SEQ ID NO:2. In some embodiments, the protein sequence retains the activity of human OX40L or activates OX40.

In some embodiments, the OX40 agonist mRNA encodes the extracellular domain of NP_003317 (i.e., residues 52-183 of SEQ ID NO:1, or a peptide having at least 70%, 75%, 80%, 85%, 90%, 95%, 98%, or 99% sequence identity to residues 52-183 of SEQ ID NO:1). In some embodiments, the OX40 agonist mRNA encodes the extracellular domain of NP 001284491 (i.e., residues 2-133 of SEQ ID NO:2, or a peptide having at least 70%, 75%, 80%, 85%, 90%, 95%, 98%, or 99% sequence identity to residues 2-133 of SEQ ID NO:2).

In some embodiments, the extracellular domain of OX40L can be fused to the transmembrane domain and optionally an intracellular fragment of another protein, such that the fusion protein can be anchored to the plasma membrane. The transmembrane domain and intracellular fragment can be from any protein, such as a human protein, in particularly those that are expressed on the membranes of cells in a tissue where OX40L is desired to be expressed.

In some embodiments, the IL-12 is human IL-12. In some embodiments, the IL-12 includes IL-12A (p35). In some embodiments, the IL-12 includes IL-12B (p40). In some embodiment, the IL-12 mRNA includes a mRNA encoding IL-12A and a mRNA encoding IL-12B. In some embodiment, the IL-12 mRNA includes a mRNA encoding both IL-12A and IL-12B.

In some embodiments, the IL-12A mRNA includes the mRNA sequence corresponding to the coding sequence of NM_000882 (SEQ ID NO:9). In some embodiments, the IL-12A mRNA encodes the protein sequence of NP_000873 (SEQ ID NO: 5), or the mature protein (residues 57-253 of SEQ ID NO:5).

In some embodiments, the IL-12A mRNA includes the mRNA sequence corresponding to the coding sequence of NM_001354582 (SEQ ID NO:10). In some embodiments, the IL-12A mRNA encodes the protein sequence of NP_001341511 (SEQ ID NO: 6), or the mature protein (residues 57-239 of SEQ ID NO:6).

In some embodiments, the IL-12A mRNA includes the mRNA sequence corresponding to the coding sequence of NM_001354583 (SEQ ID NO:11). In some embodiments, the IL-12A mRNA encodes the protein sequence of NP_001341512 (SEQ ID NO: 7), or the mature protein (residues 57-215 of SEQ ID NO:7).

In some embodiments, the IL-12A mRNA includes the mRNA sequence corresponding to the coding sequence of NM_001397992 (SEQ ID NO:12). In some embodiments, the IL-12A mRNA encodes the protein sequence of NP_001384921 (SEQ ID NO: 8), or the mature protein (residues 23-219 of SEQ ID NO:8).

In some embodiments, the IL-12B mRNA includes the mRNA sequence corresponding to the coding sequence of NM_002187 (SEQ ID NO:14). In some embodiments, the IL-12A mRNA encodes the protein sequence of NP_002178 (SEQ ID NO: 13), or the mature protein (residues 23-328 of SEQ ID NO:13).

In some embodiments, the OX40L mRNA (or mRNA encoding a protein polypeptide/antibody or their combination that activates OX40) and the IL-12 mRNA are separate mRNA molecules. In some embodiments, the OX40L (or mRNA encoding a protein polypeptide/antibody or their combination that activates OX40) coding sequence and the IL-12 coding sequence are included in the same mRNA molecule which can be translated into a fusion protein/polypeptide or separate proteins/polypeptides.

In some embodiments, one of the mRNA encodes OX40L, such as human OX40L. In some embodiments, the encoded protein has the amino acid sequence of SEQ ID NO:1. In some embodiments, the encoded protein has an amino acid sequence that has at least 70%, 80%, 85%, 90%, 95%, 98%, or 99% sequence identity to SEQ ID NO:1. In some embodiments, a fragment of the encoded protein has an amino acid sequence that has at least 90%, 95%, 98%, or 99% sequence identity to a fragment in SEQ ID NO:1. In some embodiments, the encoded protein has the amino acid sequence of SEQ ID NO:2. In some embodiments, the encoded protein has an amino acid sequence that has at least 70%, 80%, 85%, 90%, 95%, 98%, or 99% sequence identity to SEQ ID NO:2. In some embodiments, a fragment of the encoded protein has an amino acid sequence that has at least 90%, 95%, 98%, or 99% sequence identity to a fragment in SEQ ID NO:2. (Someone may make a fusion protein which contains the extracellular domain of OX40L and the intracellular and transmembrane domains from other proteins).

In some embodiments, the mRNA includes the nucleic acid sequence of SEQ ID NO: 3. In some embodiments, the mRNA includes the nucleic acid sequence that has at least 70%, 80%, 85%, 90%, 95%, 98%, or 99% sequence identity to SEQ ID NO:3. In some embodiments, the mRNA includes the nucleic acid sequence of SEQ ID NO:4. In some embodiments, the mRNA includes the nucleic acid sequence that has at least 70%, 80%, 85%, 90%, 95%, 98%, or 99% sequence identity to SEQ ID NO:4.

In some embodiments, one of the mRNA encodes IL-12A, such as human IL-12A. In some embodiments, the encoded protein has the amino acid sequence of SEQ ID NO:5. In some embodiments, the encoded protein has an amino acid sequence that has at least 70%, 80%, 85%, 90%, 95%, 98%, or 99% sequence identity to SEQ ID NO:5. In some embodiments, the encoded protein has the amino acid sequence of SEQ ID NO:6. In some embodiments, the encoded protein has an amino acid sequence that has at least 70%, 80%, 85%, 90%, 95%, 98%, or 99% sequence identity to SEQ ID NO:6. In some embodiments, the encoded protein has the amino acid sequence of SEQ ID NO:7. In some embodiments, the encoded protein has an amino acid sequence that has at least 70%, 80%, 85%, 90%, 95%, 98%, or 99% sequence identity to SEQ ID NO:7. In some embodiments, the encoded protein has the amino acid sequence of SEQ ID NO:8. In some embodiments, the encoded protein has an amino acid sequence that has at least 70%, 80%, 85%, 90%, 95%, 98%, or 99% sequence identity to SEQ ID NO:8.

In some embodiments, the mRNA includes the nucleic acid sequence of SEQ ID NO: 9. In some embodiments, the mRNA includes the nucleic acid sequence that has at least 70%, 80%, 85%, 90%, 95%, 98%, or 99% sequence identity to SEQ ID NO:9. In some embodiments, the mRNA includes the nucleic acid sequence of SEQ ID NO:10. In some embodiments, the mRNA includes the nucleic acid sequence that has at least 70%, 80%, 85%, 90%, 95%, 98%, or 99% sequence identity to SEQ ID NO: 10. In some embodiments, the mRNA includes the nucleic acid sequence of SEQ ID NO: 11. In some embodiments, the mRNA includes the nucleic acid sequence that has at least 70%, 80%, 85%, 90%, 95%, 98%, or 99% sequence identity to SEQ ID NO:11. In some embodiments, the mRNA includes the nucleic acid sequence of SEQ ID NO:12. In some embodiments, the mRNA includes the nucleic acid sequence that has at least 70%, 80%, 85%, 90%, 95%, 98%, or 99% sequence identity to SEQ ID NO: 12.

In some embodiments, one of the mRNA encodes IL-12B, such as human IL-12B. In some embodiments, the encoded protein has the amino acid sequence of SEQ ID NO:13. In some embodiments, the encoded protein has an amino acid sequence that has at least 70%, 80%, 85%, 90%, 95%, 98%, or 99% sequence identity to SEQ ID NO:13.

In some embodiments, the mRNA includes the nucleic acid sequence of SEQ ID NO: 14. In some embodiments, the mRNA includes the nucleic acid sequence that has at least 70%, 80%, 85%, 90%, 95%, 98%, or 99% sequence identity to SEQ ID NO: 14.

In some embodiments, the one or more mRNA encode, collectively OX40L (or a protein polypeptide/antibody or their combination that activates OX40) and IL-12A. In some embodiments, the one or more mRNA encode, collectively OX40L (or a protein polypeptide/antibody or their combination that activates OX40) and IL-12B. In some embodiments, the one or more mRNA encode, collectively OX40L (or a protein polypeptide/antibody or their combination that activates OX40), IL-12A and IL-12B.

When the mRNA molecules encoding IL-12 and OX40L (or a soluble counterpart) are provided on separate molecules, their ratios can be adjusted as needed. In one embodiment, the mass ratio between the IL-12 mRNA and the OX40L mRNA is from 1:0.5 to 1:6, without limitation. In one embodiment, the mass ratio between the IL-12 mRNA and the OX40L mRNA is from 1:0.5 to 1:5. In one embodiment, the mass ratio between the IL-12 mRNA and the OX40L mRNA is from 1:0.5 to 1:4. In one embodiment, the mass ratio between the IL-12 mRNA and the OX40L mRNA is from 1:0.75 to 1:6. In one embodiment, the mass ratio between the IL-12 mRNA and the OX40L mRNA is from 1:0.75 to 1:5. In one embodiment, the mass ratio between the IL-12 mRNA and the OX40L mRNA is from 1:0.75 to 1:4. In one embodiment, the mass ratio between the IL-12 mRNA and the OX40L mRNA is from 1:0.8 to 1:5. In one embodiment, the mass ratio between the IL-12 mRNA and the OX40L mRNA is from 1:0.8 to 1:4. In one embodiment, the mass ratio between the IL-12 mRNA and the OX40L mRNA is from 1:0.8 to 1:3.

In one embodiment, the mass ratio between the IL-12 mRNA and the OX40L mRNA is from 1:0.9 to 1:5. In one embodiment, the mass ratio between the IL-12 mRNA and the OX40L mRNA is from 1:0.9 to 1:4. In one embodiment, the mass ratio between the IL-12 mRNA and the OX40L mRNA is from 1:0.9 to 1:3.5. In one embodiment, the mass ratio between the IL-12 mRNA and the OX40L mRNA is from 1:0.9 to 1:3. In one embodiment, the mass ratio between the IL-12 mRNA and the OX40L mRNA is from 1:0.9 to 1:2.5.

In one embodiment, the mass ratio between the IL-12 mRNA and the OX40L mRNA is from 1:0.9 to 1:2. In one embodiment, the mass ratio between the IL-12 mRNA and the OX40L mRNA is from 1:1 to 1:4. In one embodiment, the mass ratio between the IL-12 mRNA and the OX40L mRNA is from 1:1 to 1:3. In one embodiment, the mass ratio between the IL-12 mRNA and the OX40L mRNA is from 1:1 to 1:2.5. In one embodiment, the mass ratio between the IL-12 mRNA and the OX40L mRNA is from 1:1 to 1:2.

In some embodiments, the OX40L is a full-length OX40L protein. In some embodiments, the OX40L is Fc-OX40L (soluble fragment).

As shown in the experimental examples, addition of other potential therapeutic agents may not increase the therapeutic efficacy of the instant combination of mRNA. Accordingly, in one embodiment, the treatment method or use or composition does not include some other types of mRNA.

In one embodiment, excluded are mRNAs encoding an immune checkpoint inhibitor such as PD-L1, PD-1 and CTLA-4. In one embodiment, excluded are mRNAs encoding an interferon, such as IFN-α, IFN-β, or IFN-γ. In one embodiment, excluded are mRNAs encoding another of the IL-12 family, such as IL-23, IL-27 and IL-35. In one embodiment, excluded are mRNAs encoding other cytokines, such as IL-18.

In some embodiments, each mRNA is a linear mRNA or circular mRNA. In some embodiments, each mRNA further comprises a miRNA binding site. In some embodiments, each mRNA does not include chemical modification that reduces immunogenicity. In some embodiments, the mRNA does not include chemical modification to the backbone. In some embodiments, each mRNA only includes natural nucleosides.

In some embodiments, at least one of the uridine nucleosides in the mRNAs are chemically modified. In some embodiments, the chemically modified uridine nucleosides are N1-methylpseudouridines. In some embodiments, the first mRNA and the second mRNA are formulated with a pharmaceutically acceptable carrier.

In some embodiments, the carrier comprises a lipid nanoparticle (LNP). In some embodiments, the LNP comprises (a) a molar ratio of 40-60% ionizable amino lipid, a molar ratio of 8-16% phospholipid, a molar ratio of 30-45% sterol, and a molar ratio of 1-5% PEG-modified lipid, (b) a molar ratio of 45-65% ionizable amino 40 lipid, a molar ratio of 5-10% phospholipid, a molar ratio of 25-40% sterol, and a molar ratio of 0.5-5% PEG modified lipid, (c) a molar ratio of 40-60% ionizable amino lipid, a molar ratio of 8-16% phospholipid, a molar ratio of 30-45% sterol, and a molar ratio of 1-5% PEG modified lipid, (d) a molar ratio of 45-65% ionizable amino lipid, a molar ratio of 5-10% phospholipid, a molar ratio of 25-40% sterol, and a molar ratio of 0.5-5% PEG modified lipid, (e) a molar ratio of 40-60% ionizable amino lipid, a molar ratio of 8-16% phospholipid, a molar ratio of 30-45% sterol, and a molar ratio of 1-5% PEG modified lipid, or (f) a molar ratio of 45-65% ionizable amino lipid, a molar ratio of 5-10% phospholipid, a molar ratio of 25-40% sterol, and a molar ratio of 0.5-5% PEG modified lipid.

In some embodiments, each mRNA is packaged in a liposome. In some embodiments, the liposome comprises a cationic lipid, a non-cationic lipid, a cholesterol-based lipid and a PEG modified lipid.

In some embodiments, the cationic lipid is selected from the group consisting of 1,1′-((2-(4-(2-((2-(bis(2-hydroxydodecyl)amino)ethyl) 2-hydroxydodecyl)amino)ethyl)piperazin-1-yl)ethyl)azanediyl)bis(dodecan-2-ol) (C12-200), (6Z,9Z,28Z,31Z)-heptatriaconta-6,9,28,31-tetraen-19-yl 4-(dimethylamino) butanoate (MC3), N,N-dimethyl-2,3-bis((9Z,12Z)-octadeca-9,12-dien-1-yloxy) propan-1-amine (DLinDMA), 2-(2,2-di((9Z,12Z)-octadeca-9,12-dien-1-yl)-1,3-dioxolan-4-yl)-N,N-dimethylethanamine (DLinKC2DMA, [XTC2]), 3,6-bis(4-(bis(2-hydroxydodecyl)amino)butyl)piperazine-2,5-dione (cKK-E12), 10,13-dimethyl-17-(6-methylheptan-2-yl)-2,3,4,7,8,9,10,11,12,13,14,15,16,17-tetradecahydro-1H-cyclopenta[a]phenanthren-3-yl 3-(1H-imidazol-5-yl) propanoate (ICE), (15Z,18Z)-N,N-dimethyl-6-((9Z,12Z)-octadeca-9,12-dien-1-yl)tetracosa-15,18-dien-1-amine (HGT5000), (4Z,15Z,18Z)-N,N-dimethyl-6-((9Z,12Z)-octadeca-9,12-dien-1-yl)tetracosa-4,15,18-trien-1-amine (HGT5001), N,N-dioleyl-N,N-dimethylammonium chloride (DODAC), N,N-distearyl-N,N-dimethylammonium bromide (DDAB), 1,2-dimyristyloxyproyl-3-dimethyl-hydroxyethyl ammonium bromide (DMRIE), dioleoyloxy-N-[2-sperminecarboxamido)ethyl]-N,N-dimethyl-1-propaniminiumtrifluoroacetate (DOSPA), dioctadecylamidoglycyl spermine (DOGS), 1,2-diolcoyl-3-dimethylammonium-propane (DODAP), N,N-dimethyl-(2,3-diolcyloxy) propylamine (DODMA) and N,N-dimethyl-(2,3-dimyristyloxy) propylamine (DMDMA), 1,2-dilinolenyloxy-N,N-dimethylaminopropane (DLenDMA), (2S)-2-(4-((10,13-dimethyl-17-(6-methylheptan-2-yl)-2,3,4,7,8,9,10,11,12,13,14,15,16,17-tetradecahydro-1H-cyclopenta[a]phenanthren-3-yl)oxy) butoxy)-N,N-dimethyl-3-((9Z,12Z)-octadeca-9,12-dien-1-yloxy) propan-1-amine (CLinDMA), 2-[5′-(cholest-5-en-3 [betal]-oxy)-3′-oxapentoxy)-3-dimethyl-1-1 (cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), (9Z,9′Z,12Z,12′Z)-3-(dimethylamino) propane-1,2-diyl bis(octadeca-9,12-dienoate) (DLinDAP), 1,2-dilinolcylcarbamyl-3-dimethylaminopropane (DLinCDAP), 2,2-dilinoleyl-4-dimethylaminomethyl-[1,3]-dioxolane (DLin-K-DMA), 2-((2,3-bis((9Z,12Z)-octadeca-9,12-dien-1-yloxy) propyl)disulfanyl)-N,N-dimethylethanamine (HGT4003), and

In some embodiments, the cholesterol-based lipid is cholesterol or PEGylated cholesterol. In some embodiments, the cationic lipid constitutes about 30-50% of the liposome by molar ratio. In some embodiments, the ratio of cationic lipid:non-cationic lipid:cholesterollipid:PEGylated lipid is approximately 40:30:25:5 by molar ratio. In some embodiments, the liposome comprises a combination selected from the group consisting of: cKK-E12, 1,2-dioleyl-sn-glycero-3-phosphoethanolamine (DOPE), cholesterol and 1,2-dimyristoyl-sn-glycerol, methoxypolyethylene Glycol (DMG-PEG2K); C12-200, DOPE, cholesterol and DMG-PEG2K; HGT4003, DOPE, cholesterol and DMG-PEG2K; or ICE, DOPE, cholesterol and DMG-PEG2K.

### EXAMPLES

### Example 1: COMBINATION OF IL-12 AND OX40L MRNA SYNERGISTICALLY INHIBITED TUMORS

This example evaluated the effects of various factors, delivered as synthetic mRNA, in inhibiting the growth of tumors.

The factors included a soluble PD1 (sPD1, as a PD-1/PD-L1 inhibitor), the OX40 ligand (OX40L, isoform 1), IL-12 (a IL12B-IL12A fusion protein), interferon beta (IFN-β), Resiquimod (R-848, a ligand for Toll-like receptor 7/8), and GFP as a control.

A colon cancer mouse model was used in this example. CT-26 is a murine colon cancer cell line. Eight days following implantation of CT-26 cells in mice, an mRNA sample that included individual mRNA or their mixtures (10 μg of each mRNA per mouse) (Table 1), packed in lipid nanoparticles (LNP), was injected into the tumor. At day 18, the animals were sacrificed, tumors were removed, and tumor volumes were measured. The results are shown in FIG. 1.

TABLE 1
mRNA Samples

| | mRNA #1 | | | |
|---|---|---|---|---|
| No. | (or R848) | mRNA #2 | mRNA #3 | mRNA #4 |
| 1 | GFP | — | — | — |
| 2 | GFP | GFP | — | — |
| 3 | sPD1 | — | — | — |
| 4 | OX40L | — | — | — |
| 5 | IL-12 | — | — | — |
| 6 | IFN-β | — | — | — |
| 7 | R848 | — | — | — |
| 8 | sPD1 | OX40L | — | — |
| 9 | sPD1 | IL-12 | — | — |
| 10 | OX40L | IL-12 | — | — |
| 11 | IFN-β | OX40L | — | — |
| 12 | IFN-β | IL-12 | — | — |
| 13 | IFN-β | OX40L | IL-12 | — |
| 14 | IFN-β | OX40L | sPD1 | — |
| 15 | IFN-β | sPD1 | IL-12 | OX40L |
| 16 | sPD1 | OX40L | IL-12 | — |
| 17 | R848 | sPD1 | OX40L | — |
| 18 | R848 | OX40L | IL-12 | — |
| 19 | R848 | sPD1 | IL-12 | — |
| 20 | R848 | sPD1 | OX40L | IL-12 |
| 21 | IFN-β | IL-12 | sPD1 | — |
| 22 | GFP | GFP | GFP | — |
| 23 | — | — | — | — |

Interestingly, sPD1 alone exhibited no anti-tumor effect. While OX40L showed some antitumor effects, R848 and IFN-β appeared to be more potent, and IL-12 demonstrated the most potent anti-tumor efficacy as a single agent.

Among all combination treatments, the combination of OX40L and IL-12 (Treatment No. 10) easily stood out as the most efficacious one, with a roughly 10-fold reduction of tumor size. Compared to each of the single agents (Treatment Nos. 4 and 5, respectively), this combination achieved synergistic effects.

Unexpectedly, despite its moderate anti-tumor effect as a single agent, R848 actually decreased other agents' anti-tumor effects when used in combinations. For instance, Treatment No. 18 (R848+OX40L+IL-12) was less efficacious than Treatment No. 10 (OX40L+IL-12), and Treatment No. 20 (R848+sPD1+OX40L+IL-12) was less efficacious than Treatment No. 16 (sPD1+OX40L+IL-12).

### Example 2. Anti-Tumor Effects on Non-Injected Side of Tumor

Based on the results of Example 1, this example further explored the anti-tumor efficacy of the combination mRNA treatment in distal tumor areas.

The animal model used in this example is the same as in Example 1, but the injection of the LNP-packaged OX40L and IL-12 mRNA was into one side of the tumor block. Ten days following the injections (at day 18), the tumor volumes on both sides were measured. As shown in FIG. 2, even though the mRNA was only injected to one side of the tumor block, the anti-tumor effects were substantially similar between both sides.

This example, therefore, demonstrates that the injected mRNA and/or its expressed protein product was able to spread in the tumor tissue, leading to potent inhibition of the entire tumor.

### Example 3. Anti-Tumor Efficacy Through Multiple Routes of Administration

This example further explores whether routes of administration besides intratumoral injection could also lead to effective treatment.

The same animal as in Examples 1-2 were used here. In addition to intratumoral injection, this example further included a route of subcutaneous injection (LNP-packaged OX40L and IL-12 mRNA) at a distal site from the tumor. For both routes, two injections were carried out, on day 8 and day 11 post-tumor implantation. Tumor volumes were measured three days or six days after the second injections, and the results are shown in FIG. 3.

While the intratumoral injections achieved the most profound antitumor effects, the distal subcutaneous injections were also quite efficacious.

### Example 4. Anti-Tumor Efficacy in Lung Cancer Model

This example tested the anti-tumor effects of the OX40L and IL-12 combo mRNA in a lung cancer animal model.

The lung cancer model used here was a TC-1 model. LNP-packaged OX40L and IL-12 mRNA were injected into one side of the tumor blocks on day 6 and day 9 following tumor implantation. Tumor volumes were measured on day 3 and day 6 after the treatment. The results are shown in FIG. 4.

The results show that the combination mRNA treatment was efficacious for the lung cancer model.

The distal effects of such intratumoral injections were also evaluated, as done in Example 2. The mRNA was injected to one side of the lung tumor block and the anti-tumor effects were measured for both sides. As shown in FIG. 5, the anti-tumor effects were throughout the entire tumor.

### Example 5. Anti-Tumor Effects in Triple-Negative Breast Cancer Model

This example further assessed the anti-tumor effects of the OX40L and IL-12 combo mRNA in a 4T1 animal model that modeled the triple-negative breast cancer.

Similar procedures as the preceding examples were used. As shown in FIG. 6, the OX40L and IL-12 combo mRNA almost completely inhibited tumor growth (left panel) without observable detrimental effect on the general health/body weights of the animals (right panel).

The distal effects of such intratumoral injections were also evaluated, as done in Examples 2 and 4. The mRNA was injected to one side of the breast tumor block and the anti-tumor effects were measured for both sides. As shown in FIG. 7, the anti-tumor effects were throughout the entire tumor.

### Example 6. Anti-Tumor Effects in Human mRNA in Tupaia belangeri Breast Cancer Model

This example used human OX40L and IL-12 mRNAs to treat breast cancer in Tupaia belangeri, an animal that belongs to primates.

Tumors were induced in two breasts of Tupaia belangeri. Then one side of the breast tumors were injected with human OX40L and IL-12 or GFP control mRNA/LNP nanoparticles (50 μg of each mRNA per Tupaia belangeri). Three and six days after the injection, the same side tumor was injected with the same mRNA/LNP nanoparticles for two more times. Tumor volumes in injected and un-injected sides were measured every three days till day 21.

As shown in FIG. 8, injections of the human mRNA caused suppression or even elimination of the breast tumors. Even when the mRNA was injected to one side of the breast tumor, tumors on both sides were suppressed or eliminated.

### Example 7. Comparison of Full Length OX40L and Soluble OX40L Fragment

This example compared the efficacy of full length OX40L and its soluble portion in a tumor animal model.

The test agents were produced in the animals by mRNA intratumorally injected. The agents included a control (GFP), IL-12, full-length OX40L protein, and a soluble OX40L fragment fused to an IgG Fc fragment (Fc-OX40L).

CT26 tumor cells (1×10⁶) were implanted to mice, and on days 8 and 11, respectively, test agents were injected to the animals intratumorally (1.5 μg total mRNA per animal). At time of first dosing, the CT26 tumor block was about 4 mm in diameter. The animals were inspected on days 8, 11, 14 and 17 post-implantation. As shown in FIG. 9, overall, the combination of IL-12 and OX40L (full-length or soluble) had higher tumor reduction efficacy than either agent alone, both for local tumors (“Treated”) and at the distal regions (“Distal”). Interestingly, the combination with the soluble OX40L (Fc-OX40L) outperformed the combination with the full-length OX40L.

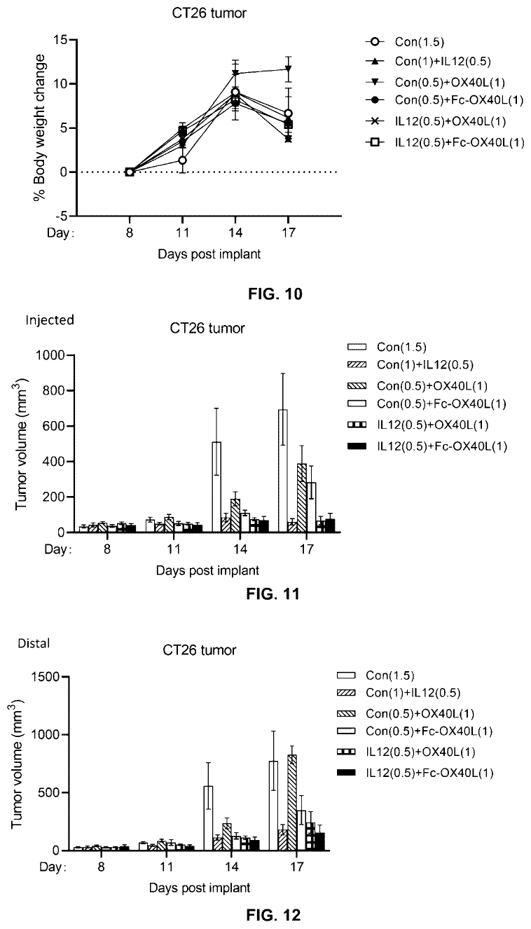

More detailed measurement data of the safety and anti-tumor efficacy of these agents are shown in FIG. 10-12, for days 8, 11, 14 and 17 post-implantation (FIG. 11, injected side; FIG. 12, distal side). All of the test agents exhibited good safety in the animals, comparable to the control (FIG. 10).

These results confirmed that the combination of IL-12 with the soluble Fc-OX40L was the most efficacious.

### Example 8. IL-12 mRNA and Anti-OX40 Antibody

This example tested whether an anti-OX40 antibody could synergize with IL-12 in inhibiting tumor growth.

The animal used here is similar to that of Example 7. On days 8 and 11 following implantation of the CT26 tumor cells, IL-12 mRNA (0.3 μg) and a test antibody (20 μg) were intratumorally injected to the animals. On day 8, the CT26 tumor blocks were about 5 mm in diameter.

The combination of IL-12 mRNA and anti-OX40 antibodies (a commercial one and a proprietary one (“HX”) achieved comparable anti-tumor results to the combination of IL-12 mRNA and anti-PD1/PD-L1 antibodies at the injection site (FIG. 13, 15). Surprisingly, at the distal side, the combinations with anti-OX40 antibodies outperformed those with anti-PD1/PD-L1 antibodies by a great margin (FIG. 13, 16).

Overall, the combination of IL-12 mRNA and anti-OX40 antibodies safely (FIG. 14) and synergistically (FIG. 13) inhibited tumor growth at both the injected side and distal side of the animals.

### Example 9. Antibody and IL-12 mRNA

This example tested whether an anti-OX40 antibody (delivered as mRNA) could synergize with IL-12 (delivered as mRNA) in inhibiting tumor growth.

The animal used here is similar to that of Example 7. On days 9 and 12 following implantation of the CT26 tumor cells, test mRNA molecules (10.5 μg in total) were intratumorally injected to the animals. On day 9, the CT26 tumor blocks were about 62 mm in diameter. The mRNA combinations are shown in FIG. 17, which included IL-12 and one of full-length OX40L, OX40 antibody, PD-antibody of PD-L1 antibody.

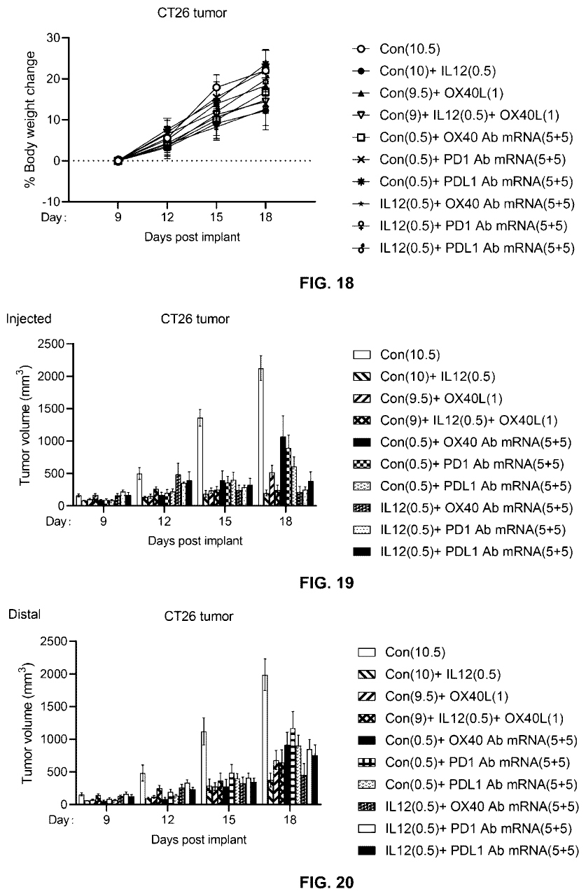

Like in Example 8, the combination of IL-12 with OX40L/OX40 antibody achieved the best anti-tumor efficacy (FIG. 17, 19) in particular at the distal side (FIG. 17, 20). All the test agents were safe in the animals (FIG. 18).

### Example 10. IL-12, OX12L and GM-CSF

This example tested whether adding additional agents could further increase the anti-tumor efficacy of the IL-12/OX40L combination.

Three additional agents were tested, including GM-CSF (Granulocyte-macrophage colony-stimulating factor), GSDMD (Gasdermin D), and TNFR (tumor necrosis factor receptor). GM-CSF stimulates monocytes and macrophages to produce pro-inflammatory cytokines. GSDMD serves as a specific substrate of inflammatory caspases (caspase-1,-4,-5 and -11) and as an effector molecule for the lytic and highly inflammatory form of programmed cell death, pyroptosis.

All of these agents were delivered, in various combinations, to the same animal model as used above, as mRNA (1.5 μg in total), on days 9 and 12, post-tumor cell implantation. While IL-12 and OX40L were encoded on different constructs, the added agent (e.g., GM-CSF) was fused to the OX40L mRNA, through an IRES linker.

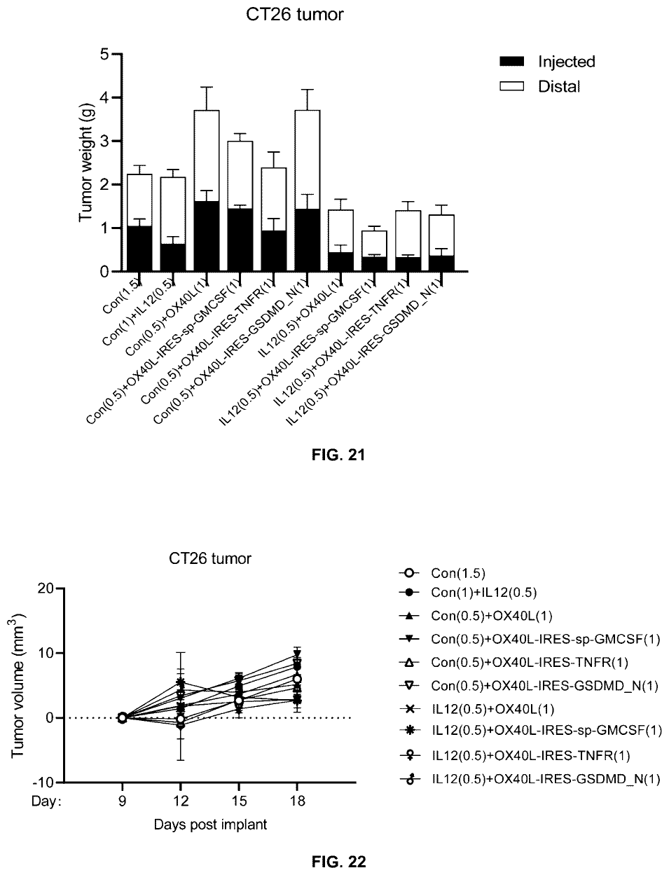

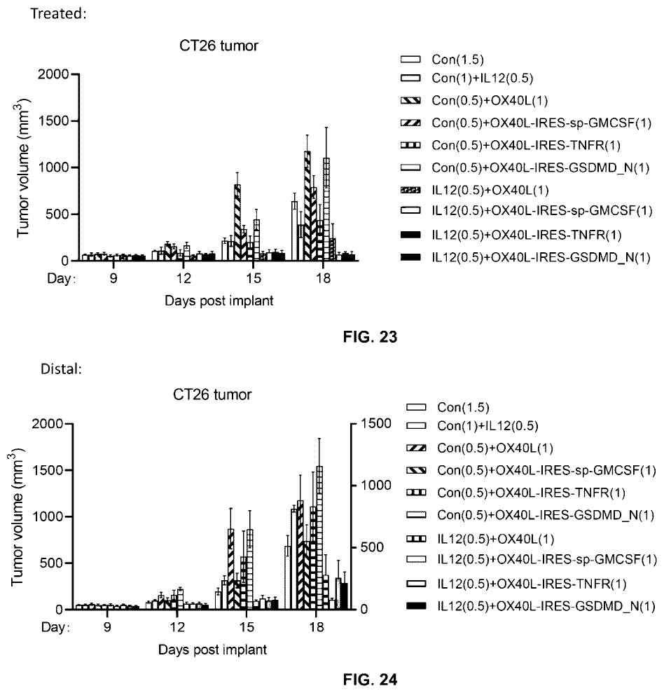

As shown in FIG. 21, the addition of each of GM-CSF, GSDMD and TNER further increased the efficacy of the IL-12/OX12L combination. However, the tri-member combination with GM-CSF achieved the highest efficacy (FIG. 21, 23). In particular, the IL-12/OX40L/GM-CSF combination exhibited remarkably higher efficacy at the distal side of the animals (FIG. 21, 24). All the test agents were safe in the animals (FIG. 22).

### Example 11. IL-12 and OX40L Ratio

This example tested whether different ratios of IL-12 mRNA to OX40L could lead to different results.

Each animal, on days 8 and 11 post-implantation, received a total of 4.3 μg mRNA as indicated in FIG. 25 (mRNA expressing GFP was added, where needed, to ensure the total amount of mRNA being delivered was 4.3 μg). At about 1:1 to 1:3 ratio, the combination of IL-12 mRNA and OX40L mRNA exhibited the highest tumor growth inhibition efficacy (FIG. 25, 27, 28), at both the injected and distal sides.

While IL-12 alone at higher doses (e.g., 1 μg and 2 μg vs. 0.3 μg in the combinations) appeared to have higher rate of tumor inhibition, it also had the highest negative impact on body weight growth (FIG. 26), suggesting that these higher doses were associated with toxicities. Therefore, the combination between IL-12 and OX40L not only increased tumor inhibition efficacy, but also led to improved safety.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for treating cancer in a patient, comprising administering to the patient a first mRNA encoding an OX40 agonist, and a second mRNA encoding IL-12, wherein the OX40 agonist is an OX40 ligand (OX40L), a polypeptide comprising the extracellular domain of OX40L, or an agonist anti-OX40 antibody or antigen-binding fragment thereof.

2. The method of claim 1, wherein the first mRNA and the second mRNA are included in the same RNA molecule.

3. The method of claim 2, wherein the RNA molecule encodes a polypeptide that comprises the OX40 agonist and IL-12 as a fusion protein, or encodes separate polypeptides.

4. The method of claim 1, wherein the first mRNA and the second mRNA are separate molecules.

5. The method of claim 1, wherein the OX40 agonist is the OX40L.

6. The method of claim 5, wherein the OX40L comprises the amino acid sequence of SEQ ID NO:1 or 2, or an amino acid sequence having at least 85% sequence identity to SEQ ID NO: 1 or 2.

7. The method of claim 5, wherein the first mRNA comprises the nucleic acid sequence of SEQ ID NO:3 or 4, or a nucleic acid sequence having at least 85% sequence identity to SEQ ID NO:3 or 4.

8. The method of claim 1, wherein the extracellular domain of OX40L comprises amino acid residues 52-183 of SEQ ID NO:1, or a sequence having at least 85% sequence identity to amino acid residues 52-183 of SEQ ID NO:1.

9. The method of claim 1, wherein the polypeptide further comprises an oligomerization domain or a transmembrane domain.

10. The method of claim 9, wherein the OX40 agonist comprises a Fc domain fused to the extracellular domain of OX40L, and wherein the OX40 agonist is a soluble protein not containing the transmembrane domain of the OX40L protein.

11. The method of claim 1, wherein the second mRNA encodes IL-12A.

12. The method of claim 11, wherein the IL-12A comprises an amino acid sequence selected from the group consisting of residues 57-253 of SEQ ID NO:5, residues 57-239 of SEQ ID NO: 6, residues 57-215 of SEQ ID NO:7 and residues 23-219 of SEQ ID NO:8, or an amino acid having at least 85% sequence identity to any amino acid sequence of the group.

13. The method of claim 11, wherein the second mRNA comprises the nucleic acid sequence of SEQ ID NO:9, 10, 11 or 12, or a nucleic acid sequence having at least 85% sequence identity to SEQ ID NO:9, 10, 11 or 12.

14. The method of claim 12, further comprising administering to the patient a third mRNA, wherein the third mRNA encodes residues 23-328 of SEQ ID NO:13.

15. The method of claim 1, wherein the second mRNA encodes IL-12B.

16. The method of claim 15, wherein the IL-12B comprises residues 23-328 of SEQ ID NO: 13, or an amino acid sequence having at least 85% sequence identify to residues 23-328 SEQ ID NO:13.

17. The method of claim 15, wherein the second mRNA comprises the nucleic acid sequence of SEQ ID NO:14, or a nucleic acid sequence having at least 85% sequence identity to SEQ ID NO:14.

18-20. (canceled)

21. The method of claim 1, further comprising administering to the patient a fourth mRNA encoding GM-CSF (granulocyte-macrophage colony-stimulating factor).

22-23. (canceled)

24. A method for treating cancer in a patient, comprising administering to the patient a first agent comprising a mRNA encoding IL-12, and a second agent comprising an OX40 agonist, wherein the OX40 agonist is an agonist anti-OX40 antibody or antigen-binding fragment thereof, an OX40 ligand (OX40L), or a polypeptide comprising the extracellular domain of OX40L.

25-46. (canceled)

47. A pharmaceutical composition comprising a first agent comprising a mRNA encoding IL-12, and a second agent comprising an OX40 agonist, wherein the OX40 agonist is an agonist anti-OX40 antibody or antigen-binding fragment thereof, an OX40 ligand (OX40L), or a polypeptide comprising the extracellular domain of OX40L.


SEQUENCE LISTING (16 sequences)

SEQ ID NO: 1 - protein, 183 aa, synthetic construct
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 2 - protein, 133 aa, synthetic construct
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3 - RNA, 552 nt, synthetic construct
  source 1..552: mol_type = other RNA
atggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 4 - RNA, 402 nt, synthetic construct
  source 1..402: mol_type = other RNA
atggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 5 - protein, 253 aa, synthetic construct
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 239 aa, synthetic construct
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - protein, 215 aa, synthetic construct
MWPPGSASQPPPSPAAATGLHPAARPVSLQCRLSMCPARSLLLVATLVLLDHLSLARNLPVATPDPGMFPCLHHSQNLLRAVSNMLQKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - protein, 219 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 9 - RNA, 762 nt, synthetic construct
  source 1..762: mol_type = other RNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 10 - RNA, 720 nt, synthetic construct
  source 1..720: mol_type = other RNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 11 - RNA, 648 nt, synthetic construct
  source 1..648: mol_type = other RNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 12 - RNA, 660 nt, synthetic construct
  source 1..660: mol_type = other RNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 13 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 14 - RNA, 986 nt, synthetic construct
  source 1..986: mol_type = other RNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtta

SEQ ID NO: 15 - protein, 144 aa, synthetic construct
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 16 - RNA, 435 nt, synthetic construct
  source 1..435: mol_type = other RNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga